(19) United States
(12) Patent Application Publication
(10) Pub. No.: US 2023/0064744 A1
(43) Pub. Date: Mar. 2, 2023

(54) Augmented Analyte Monitoring System

(71) Applicant: Dexcom, Inc., San Diego, CA (US)

(72) Inventors: Samuel Isaac Epstein, Chevy Chase, MD (US); Devon Headen, San Diego, CA (US); Avid Najdahmadi, San Diego, CA (US); Joseph J Baker, San Diego, CA (US); Kevin Cheng, San Diego, CA (US); Nicholas Vincent Apollo, San Diego, CA (US); Berta Esteban Fernandez de Avila, San Diego, CA (US); Daud Abd al-Malik Zoss, San Diego, CA (US); Wenjie Lan, Westford, MA (US)

(73) Assignee: Dexcom, Inc., San Diego, CA (US)

(21) Appl. No.: 17/899,135

(22) Filed: Aug. 30, 2022

Related U.S. Application Data
(60) Provisional application No. 63/239,811, filed on Sep. 1, 2021.

(51) Int. Cl. A61B 5/00; A61B 5/369

(52) U.S. Cl. CPC A61B 5/0006; A61B 5/369

(57) ABSTRACT

An augmented analyte monitoring system is described. The augmented analyte monitoring system includes a wearable analyte monitoring device that includes a transmitter and an analyte sensor to obtain analyte data of a user, and an analyte augmentation wearable that includes one or more sensors (e.g., physical and/or biochemical sensors) to obtain additional physiological data for augmenting the analyte data of the user. The analyte augmentation wearable is communicably coupled to the wearable analyte monitoring device. The augmented analyte monitoring system further includes a sensor hub implemented at a computing device to obtain a data packet containing both the analyte data and the additional physiological data from at least one of the wearable analyte monitoring device or the analyte augmentation wearable, and augment the analyte data by storing the analyte data in association with the additional physiological data.

### RELATED APPLICATION

This application claims the benefit of U.S. Provisional Patent Application No. 63/239,811 filed Sep. 1, 2021, and titled “Augmented Analyte Monitoring System,” the entire disclosure of which is hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating, exercising, sleep, and stress. Advances in medical technologies have enabled development of various systems for monitoring blood glucose, including continuous glucose monitoring (CGM) systems, which measure and record glucose concentrations in substantially real-time. CGM systems are important tools that help users maintain their measured glucose values within the acceptable range.

Analyte monitoring systems, such as continuous glucose monitoring systems, can be configured as wearable devices, which include sensors that can be inserted into the skin of a user to monitor an analyte, e.g., glucose. Such analyte monitoring systems can also be communicably coupled to user devices (e.g., a user's smartphone) so that data describing the analyte can be transmitted to a user device and output to the user, e.g., via a user interface. Some users and healthcare providers would like to collect other sensor data to augment the analyte data collected by the analyte monitoring system, e.g., to provide additional context to the analyte data, enable the generation of various insights regarding the analyte data, confirm whether candidates for events identified from the analyte data actually occurred, and so forth. However, conventional analyte monitoring systems are generally limited to monitoring a single analyte and/or limited analytes and physiological signals and are thus unable to augment the analyte data with diverse data describing different analytes and/or signals. Moreover, adding additional sensors to an analyte monitoring device in order to sense data for additional analytes and/or signals may increase the complexity and size of the device, while also requiring additional resources (e.g., processing and/or battery resources) to be added to the device.

### SUMMARY

To overcome these problems, an augmented analyte monitoring system is leveraged. The augmented analyte monitoring system includes a wearable analyte monitoring device that includes a transmitter and an analyte sensor to obtain analyte data of a user. The augmented analyte monitoring system also includes an analyte augmentation wearable that includes one or more sensors to obtain additional physiological data for augmenting the analyte data of the user. The analyte augmentation wearable is communicably coupled to the wearable analyte monitoring device via a communicative coupling. The augmented analyte monitoring system further includes a sensor hub implemented at a computing device to obtain a data packet containing both the analyte data and the additional physiological data from at least one of the wearable analyte monitoring device or the analyte augmentation wearable, and augment the analyte data by storing the analyte data in association with the additional physiological data.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an exemplary implementation that is operable to employ techniques described herein.

FIG. 2 depicts an example of a wearable analyte monitoring device in greater detail.

FIG. 3 depicts an example of an implementation of augmenting analyte data from a wearable analyte monitoring device with additional physiological data from an analyte augmentation wearable.

FIG. 4 depicts an example of a first different implementation of augmenting analyte data from the wearable analyte monitoring device with additional physiological data from the analyte augmentation wearable.

FIG. 5 depicts an example of a second different implementation of augmenting analyte data from the wearable analyte monitoring device with additional physiological data from the analyte augmentation wearable.

FIG. 6 depicts an example of an implementation of an analyte augmentation wearable configured as an underlay to augment the wearable analyte monitoring device.

FIG. 7 depicts an example of an implementation of an analyte augmentation wearable configured as an overlay to augment the wearable analyte monitoring device.

FIG. 8 depicts an example of an implementation of an analyte augmentation wearable configured as an overlay with a satellite extension to augment the wearable analyte monitoring device.

FIG. 9 depicts an example of an implementation of a user interface of a computing device displaying both analyte data obtained from a wearable analyte monitoring device and additional physiological data obtained from an analyte augmentation wearable.

FIG. 10 depicts a procedure in an example implementation in which a wearable analyte monitoring device generates a data packet containing both analyte data and additional physiological data and communicates the data packet to a sensor hub.

FIG. 11 depicts a procedure in an example implementation in which an analyte augmentation wearable generates a data packet containing both analyte data and additional physiological data and communicates the data packet to a sensor hub.

FIG. 12 depicts an example of the augmented analyte monitoring system that includes an analyte augmentation wearable configured for optical sensing techniques.

FIG. 13 depicts an example of the augmented analyte monitoring system that includes an analyte augmentation wearable configured as an underlay for optical sensing techniques.

FIG. 14 depicts an example of the augmented analyte monitoring system that includes an analyte augmentation wearable configured as an overlay for optical sensing techniques.

FIG. 15 illustrates an example of a system including various components of an example device that can be implemented as any type of computing device as described and/or utilized with reference to FIGS. 1-14 to implement embodiments of the techniques described herein.

### DETAILED DESCRIPTION

Overview

An augmented analyte monitoring system is described. In accordance with the described techniques, the augmented analyte monitoring system includes a wearable analyte monitoring device and an analyte augmentation wearable. The wearable analyte monitoring device is configured to provide measurements of an analyte of a person, e.g., a person's glucose. For example, the wearable analyte monitoring device may be configured with a sensor that detects one or more signals indicative of a level of the analyte in the person and enables generation of analyte measurements. Those analyte measurements may correspond to or otherwise be packaged for communication to a computing device as analyte data. In one or more implementations, for instance, the analyte monitoring device may be a wearable glucose monitoring device to generate glucose measurements indicating the person's glucose and package those measurements as glucose data.

The analyte augmentation wearable is configured to provide information describing one or more additional analytes and/or physiological signals of the person that are different from the analyte monitored by the wearable analyte monitoring device. Examples of such information include, for instance, measurements of one or more different analytes, measurements of various detected signals (e.g., biopotential measurements such as electrocardiogram (ECG), electromyography (EMG), or electroencephalogram (EEG); acceleration experienced by the person at a location where the analyte augmentation wearable is worn; and optical signals such as photoplethysmogram (PPG) that detect changes in blood volume), measurements of various physiological conditions (e.g., perspiration, temperature, heart rate, oxygen saturation (SpO₂)), or indications of detected events (e.g., exceeding or falling below a threshold, detecting the presence or absence of a particular compound), to name just a few. The analyte augmentation wearable may be configured with one or more sensors to provide information about the person that augments the analyte measurements produced by the analyte monitoring system. For example, the analyte augmentation wearable may be configured with a single or multi-analyte sensing architecture to provide the additional information. This additional information, that augments those analyte measurements and that is produced by the analyte augmentation wearable, may correspond to, or otherwise be packaged for, communication to the computing device as additional physiological data.

Notably, the analyte augmentation wearable is a specialized device that is separate from the wearable analyte monitoring device and specifically configured to augment or extend the functionality of the wearable analyte monitoring device. Moreover, in one or more implementations, the analyte augmentation wearable has a form factor that is complementary with a form factor of the wearable analyte monitoring device. By way of example, the analyte augmentation wearable may be configured as an underlay patch with one or more sensors, e.g., to produce the additional physiological data. In configurations as an underlay patch, the analyte augmentation wearable may be configured to be disposed at least partially between the wearable analyte monitoring device and the skin of the person when deployed. In one example of this configuration, the analyte augmentation wearable may be applied to the person's skin, and then the wearable augmentation monitoring device may be applied “on top” of the already applied analyte augmentation wearable.

Alternatively, the analyte augmentation wearable may be configured as an overlay patch with one or more sensors, e.g., to produce the additional physiological data. In configurations as an overlay patch, the analyte augmentation wearable may be configured to be disposed at least partially covering the wearable analyte monitoring device, such that when deployed the wearable analyte monitoring device is disposed at least partially between the analyte augmentation wearable and the skin of the person. In one example of this configuration, the wearable analyte monitoring device may be applied to the person's skin, and then the analyte augmentation wearable may be applied “on top” of the already applied wearable analyte monitoring device.

Regardless of the way in which the analyte augmentation wearable is disposed relative to the wearable analyte monitoring device, the wearable analyte monitoring device and the analyte augmentation wearable may be communicably coupled to each other. The coupling between the wearable analyte monitoring device and the analyte augmentation wearable may be configured as a “wired” or “wireless” coupling. In one or more implementations, this coupling enables the analyte augmentation wearable to communicate the additional physiological data to the wearable analyte monitoring device. In this scenario, the wearable analyte monitoring device is configured to generate a data packet containing both the additional physiological data provided by the analyte augmentation wearable as well as the analyte data produced using the sensor of the analyte monitoring device. A transmitter of the wearable analyte monitoring device then communicates the data packet containing both the additional physiological data and the analyte data to a sensor hub that is implemented at the computing device. Communicating both the analyte data and the additional physiological data in a single data packet reduces the need for the analyte augmentation wearable to have a transmitter capable of communicating data to the sensor hub, while also reducing the number of data transmissions required for the sensor hub to obtain the analyte data and the additional physiological data.

Alternatively, the communicative coupling may enable the analyte monitoring device to communicate the analyte data to the analyte augmentation wearable. In this scenario, the analyte augmentation wearable can generate a data packet containing both the additional physiological data produced using one or more sensors of the analyte augmentation wearable as well as the analyte data provided by the analyte monitoring device. A transmitter of the analyte augmentation wearable then communicates the data packet containing both the additional physiological data and the analyte data to the sensor hub that is implemented at the computing device.

The sensor hub may be configured to receive the data packet from the augmented analyte monitoring system, e.g., via the analyte monitoring device or via the augmented analyte wearable in other implementations. The sensor hub parses the augmented analyte packet and augments the analyte data by storing the analyte data in association with the additional physiological data in a storage device. In other words, the sensor hub may modify the augmented analyte packet for storage and/or extract the analyte data and the additional physiological data from the augmented analyte packet and store the extracted data with associated data, such as by associating time stamps with the extracted data, performing computations on some of the data (e.g., computing statistics on some of the data and storing the computed statistics with the data), interpolating missing data, identifying erroneous data, and so forth. This augmented analyte data may then be used in connection with one or more services provided to the user, such as by displaying the additional physiological data in a user interface along with the analyte data, generating and outputting one or more insights about the user's health based on both the analyte data and the additional physiological data, confirming whether candidates for events identified from the analyte data actually occurred, generating and outputting insights related to one or more conditions (e.g., diabetes, heart disease, etc.) for which the analyte data may also be collected, generating and outputting insights in relation to one or more conditions that are complementary to insights derived from the analyte data for those conditions, and so forth.

Thus, unlike conventional analyte monitoring systems, the described augmented analyte monitoring system is able to augment analyte data with additional physiological data to generate information and/or content that is more robust (e.g., accurate or actionable) than when the analyte data is not augmented with the additional physiological data. The analyte data and additional physiological data not only enable generation of information and/or content that is more robust than conventional techniques, but also enable the generation of different measurements than conventional techniques, e.g., due to the architecture of the augmented analyte monitoring system that combines production and communication of the analyte data and the additional physiological data. In particular, these different measurements may be generated based on covariance of the analyte and additional physiological signals, as produced using the architecture of the augmented analyte monitoring system that combines the detection of those signals and generation of corresponding measurements. Examples of the information that may be generated using the augmented analyte data and which may be more accurate and/or actionable due to using the augmented analyte data include, for instance, reports, user interfaces that plot estimated values as received, notifications of events (or reduction of notifications of events), and notifications of predicted events (or reduction of notifications about predicted events), to name just a few. One example of a different measurement that may be produced using the covariance of the analyte and additional physiological signals is an early detection of sepsis—a potentially life-threatening condition that occurs when a body's response to an infection damages its own tissues—which is not determinable solely from lactate data. Instead, sepsis may be detected earlier using the augmented analyte monitoring system and by determining a covariance of lactate with heart rate variability (HRV), blood pressure, and temperature. Additional physiological data that may be used to determine a metric for sepsis detection (e.g., a sepsis deterioration risk metric) may include movement (e.g., acceleration) detected using an accelerometer, for instance.

Moreover, the analyte augmentation wearable can augment the analyte monitoring device in a variety of different ways other than just by the type of data that is collected. For example, in one or more implementations, the analyte augmentation wearable may be configured to share various components or resources with the wearable analyte monitoring device. By way of example, the analyte augmentation wearable may share battery power with the analyte monitoring device thereby extending the operating life of the analyte monitoring device. Alternatively or additionally, the analyte augmentation wearable may leverage resources of the analyte monitoring device, such as by using at least a portion of a battery or transmission architecture of the analyte monitoring device.

In some cases, the analyte augmentation wearable may be configured in a variety of different models, each of which may include different sensors or architectures for sensing different analytes and/or physiological signals from the wearable analyte monitoring device. This enables users and healthcare providers to select an appropriate analyte augmentation wearable for a user based on the user's health condition. In other words, different analyte augmentation wearables can be combined with the wearable analyte monitoring device to form systems for producing numerous combinations of analyte data and additional physiological data. Notably, simply adding a plurality of different sensors to the wearable analyte monitoring device would greatly increase the engineering complexity of the wearable analyte monitoring device, increase the processing and battery resources required by the wearable analyte monitoring device, and so forth.

Thus utilizing the analyte augmentation wearable—which in some instances can be configured in different ways with different types of sensors and/or architectures to produce additional physiological data—enables analyte data to be augmented without the need to generally modify the wearable analyte monitoring device itself. By combining an analyte monitoring device with one or more analyte augmentation wearables, for example, the combined architecture may be used to produce data describing a person's uric acid and movement (e.g., acceleration) along with heart rate and oxygen saturation (SpO₂). For instance, the analyte monitoring device may be configured with one or more sensors to provide measurements of a person's uric acid, and the analyte augmentation wearable may be configured with one or more different sensors from the analyte monitoring device. By way of example, the analyte augmentation wearable may be configured with an accelerometer to produce data describing movement of the person and also configured with one or more sensors for producing PPG data, from which heart rate of the person and SpO₂ can be derived.

In some aspects, the techniques described herein relate to a system including: a wearable analyte monitoring device including a transmitter and an analyte sensor to obtain analyte data of a user; an analyte augmentation wearable including one or more sensors to obtain additional physiological data for augmenting the analyte data of the user, the analyte augmentation wearable communicably coupled to the wearable analyte monitoring device via a wired or wireless connection; and a sensor hub implemented at a computing device to obtain a data packet containing both the analyte data and the additional physiological data from at least one of the wearable analyte monitoring device or the analyte augmentation wearable, and augment the analyte data by storing the analyte data in association with the additional physiological data.

In some aspects, the techniques described herein relate to a system, wherein the additional physiological data describes at least one of an additional analyte of the user or one or more physiological signals of the user.

In some aspects, the techniques described herein relate to a system, wherein the analyte augmentation wearable has a first form factor that is complementary to a second form factor of the wearable analyte monitoring device.

In some aspects, the techniques described herein relate to a system, wherein the analyte augmentation wearable includes at least one of: an access that allows the analyte sensor of the wearable analyte monitoring device to pass through the access and into skin of the user; an access that fits around the wearable analyte monitoring device such that the analyte augmentation wearable can be applied to skin of the user around the wearable analyte monitoring device; a cavity having a complementary shape to the wearable analyte monitoring device such that the wearable analyte monitoring device fits within the cavity of the analyte augmentation wearable and is covered when applied to the skin of the user; or a partial cavity having a complementary shape to the wearable analyte monitoring device such that a portion of the wearable analyte monitoring device fits within the partial cavity of the analyte augmentation wearable and such that the portion of the wearable analyte monitoring device is covered when applied while another portion of the wearable analyte monitoring device is exposed.

In some aspects, the techniques described herein relate to a system, wherein the analyte augmentation wearable includes one or more components that physically contact at least a portion of the wearable analyte monitoring device when the analyte augmentation wearable and the wearable analyte monitoring device are worn by the user.

In some aspects, the techniques described herein relate to a system, wherein the analyte augmentation wearable includes an underlay patch that is configured to be disposed at least partially between the wearable analyte monitoring device and skin of the user.

In some aspects, the techniques described herein relate to a system, wherein the analyte augmentation wearable includes an overlay patch, and wherein the wearable analyte monitoring device is configured to be disposed at least partially between the analyte augmentation wearable and skin of the user.

In some aspects, the techniques described herein relate to a system, wherein the analyte augmentation wearable includes an overlay patch with a satellite extension, and wherein the satellite extension is configured to position the one or more sensors of the analyte augmentation wearable at least a threshold distance away from the wearable analyte monitoring device.

In some aspects, the techniques described herein relate to a system, wherein the wearable analyte monitoring device is further configured to: obtain the additional physiological data from the analyte augmentation wearable via the wired or wireless connection; form the data packet containing both the analyte data and the additional physiological data; and transmit the data packet containing both the analyte data and the additional physiological data to the sensor hub using the transmitter.

In some aspects, the techniques described herein relate to a system, wherein the analyte augmentation wearable is further configured to compress the additional physiological data and transmit compressed additional physiological data to the wearable analyte monitoring device.

In some aspects, the techniques described herein relate to a system, wherein the wearable analyte monitoring device is further configured to transmit the analyte data to the analyte augmentation wearable using the transmitter.

In some aspects, the techniques described herein relate to a system, wherein the analyte augmentation wearable is further configured to: obtain the analyte data from the wearable analyte monitoring device via the wired or wireless connection; form the data packet containing both the analyte data and the additional physiological data; and transmit the data packet containing both the analyte data and the additional physiological data to the sensor hub.

In some aspects, the techniques described herein relate to a system, wherein the wearable analyte monitoring device is further configured to compress the analyte data and transmit compressed analyte data to the analyte augmentation wearable.

In some aspects, the techniques described herein relate to a computer-implemented method including: obtaining analyte data of a user by an analyte sensor of a wearable analyte monitoring device worn by the user; obtaining additional physiological data for augmenting the analyte data of the user by one or more sensors of an analyte augmentation wearable, the analyte augmentation wearable communicably coupled to the wearable analyte monitoring device via a wired or wireless connection; obtaining, by a sensor hub implemented at a computing device, a data packet containing both the analyte data and the additional physiological data from at least one of the wearable analyte monitoring device or the analyte augmentation wearable; and augmenting the analyte data by storing the analyte data in association with the additional physiological data.

In some aspects, the techniques described herein relate to a computer-implemented method, wherein the analyte augmentation wearable has a first form factor that is complementary to a second form factor of the wearable analyte monitoring device.

In some aspects, the techniques described herein relate to a computer-implemented method, further including: obtaining, by the wearable analyte monitoring device, the additional physiological data from the analyte augmentation wearable via the wired or wireless connection; forming the data packet containing both the analyte data and the additional physiological data; and transmitting the data packet containing both the analyte data and the additional physiological data to the sensor hub using a transmitter of the wearable analyte monitoring device.

In some aspects, the techniques described herein relate to a computer-implemented method, further including: obtaining, by the analyte augmentation wearable, the analyte data from the wearable analyte monitoring device via the wired or wireless connection; forming the data packet containing both the analyte data and the additional physiological data; and transmit the data packet containing both the analyte data and the additional physiological data to the sensor hub using a transmitter of the analyte augmentation wearable.

In some aspects, the techniques described herein relate to a method implemented by a wearable analyte monitoring device worn by a user, the method including: establishing a first wired or wireless connection with a sensor hub implemented at a computing device associated with the user and establishing a second wired or wireless connection with an analyte augmentation wearable worn by the user; collecting analyte data of the user via an analyte sensor of the wearable analyte monitoring device worn by the user; obtaining additional physiological data from the analyte augmentation wearable worn by the user via the second wired or wireless connection; packaging the analyte data collected by the analyte sensor of the wearable analyte monitoring device with the additional physiological data obtained from the analyte augmentation wearable to form an augmented analyte packet; and communicating the augmented analyte packet containing both the analyte data collected by the analyte sensor of the wearable analyte monitoring device and the additional physiological data obtained from the analyte augmentation wearable to the sensor hub via the first wired or wireless connection.

In some aspects, the techniques described herein relate to a method, wherein the communicating further includes communicating the augmented analyte packet containing both the analyte data and the additional physiological data to the sensor hub at predefined intervals.

In some aspects, the techniques described herein relate to a method implemented by an analyte augmentation wearable worn by a user, the method including: establishing a first wired or wireless connection with a sensor hub implemented at a computing device associated with the user and establishing a second wired or wireless connection with a wearable analyte monitoring device worn by the user; obtaining analyte data from the wearable analyte monitoring device worn by the user via the second wired or wireless connection; collecting additional physiological data of the user via one or more sensors of the analyte augmentation wearable worn by the user; packaging the analyte data obtained from the wearable analyte monitoring device with the additional physiological data collected by the one or more sensors of the analyte augmentation wearable worn by the user to form an augmented analyte packet; and communicating the augmented analyte packet containing both the analyte data obtained from the wearable analyte monitoring device and the additional physiological data collected by the one or more sensors of the analyte augmentation wearable to the sensor hub via the first wired or wireless connection.

In some aspects, the techniques described herein relate to a method, wherein the communicating further includes communicating the augmented analyte packet containing both the analyte data and the additional physiological data to the sensor hub at predefined intervals.

In some aspects, the techniques described herein relate to an apparatus including: one or more sensors to collect physiological data of a user; and an underlay patch configured to directly contact skin of the user, the underlay patch including an access portion; wherein a wearable analyte monitoring device is configured to be disposed on top of the underlay patch, and wherein the access portion of the underlay patch enables an analyte sensor of the wearable analyte monitoring device to extend through the access portion of the underlay patch and insert subcutaneously into the skin of the user to collect analyte data of the user.

In some aspects, the techniques described herein relate to an apparatus, wherein the one or more sensors include at least one of electrodes or photonics.

In some aspects, the techniques described herein relate to an apparatus, wherein the apparatus is configured to communicate the physiological data of the user to the wearable analyte monitoring device via a wired or wireless connection with the wearable analyte monitoring device.

In some aspects, the techniques described herein relate to an apparatus including: one or more sensors to collect physiological data of a user; and an overlay patch configured to be applied on top of a wearable analyte monitoring device worn by the user, wherein the overlay patch includes an adhesive for adhering the overlay patch to the wearable analyte monitoring device and skin of the user and wherein the overlay patch has a geometry that is complementary with a form factor of the wearable analyte monitoring device.

In some aspects, the techniques described herein relate to an apparatus, wherein the overlay patch causes the one or more sensors to be deployed within a threshold distance of an analyte sensor of the wearable analyte monitoring device.

In some aspects, the techniques described herein relate to an apparatus including: one or more sensors to collect physiological data of a user; and an overlay patch configured to be applied on top of a wearable analyte monitoring device worn by the user, the overlay patch including a satellite extension to position the one or more sensors at least a threshold distance away from an analyte sensor of the wearable analyte monitoring device.

In some aspects, the techniques described herein relate to an apparatus, wherein the overlay patch further includes an adhesive for adhering the overlay patch to the wearable analyte monitoring device and skin of the user.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

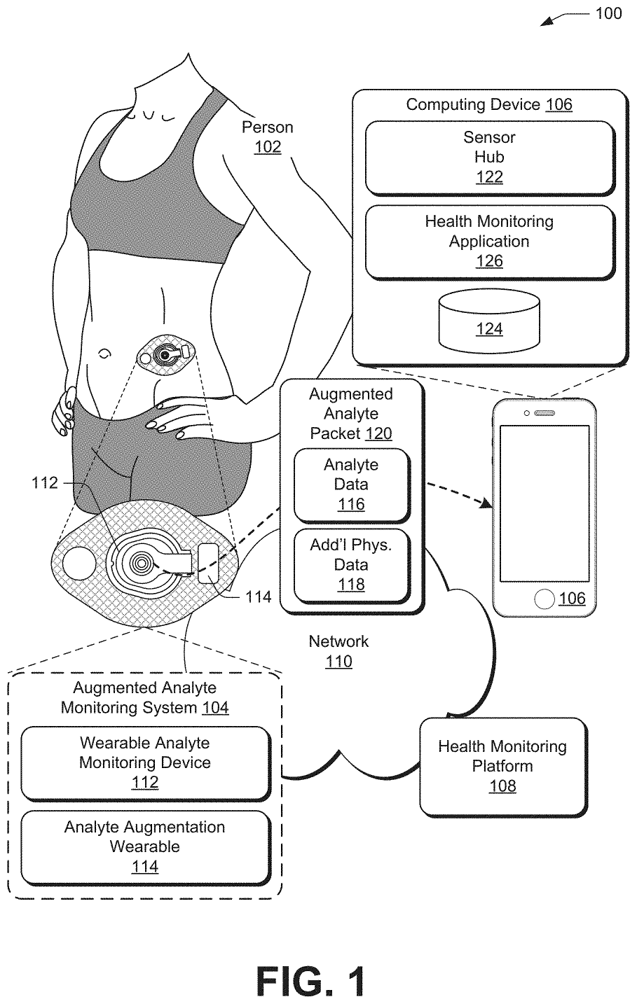

FIG. 1 is an illustration of an environment 100 in an example implementation that is operable to employ an augmented analyte monitoring system as described herein. The illustrated environment 100 includes person 102, who is depicted wearing an augmented analyte monitoring system 104. The illustrated environment 100 also includes computing device 106 and health monitoring platform 108. The augmented analyte monitoring system 104, the computing device 106, and the health monitoring platform 108 are communicably coupled, including via network 110.

The augmented analyte monitoring system 104 and the computing device 106 may be communicably coupled in various ways, such as by using one or more wireless communication protocols or techniques. By way of example, the augmented analyte monitoring system 104 and the computing device 106 may communicate with one another using one or more of radio, cellular, Wi-Fi, Bluetooth (e.g., Bluetooth Low Energy links), near-field communication (NFC), 5G, and so forth.

In accordance with the described techniques, the augmented analyte monitoring system 104 includes a wearable analyte monitoring device 112 and an analyte augmentation wearable 114. The wearable analyte monitoring device 112 is configured to provide measurements of an analyte of the person 102, e.g., the person 102's glucose. For example, the wearable analyte monitoring device 112 may be configured with an analyte sensor that detects one or more signals indicative of the analyte in the person 102 and enables generation of analyte measurements. Those analyte measurements may correspond to or otherwise be packaged for communication to the computing device 106 as analyte data 116.

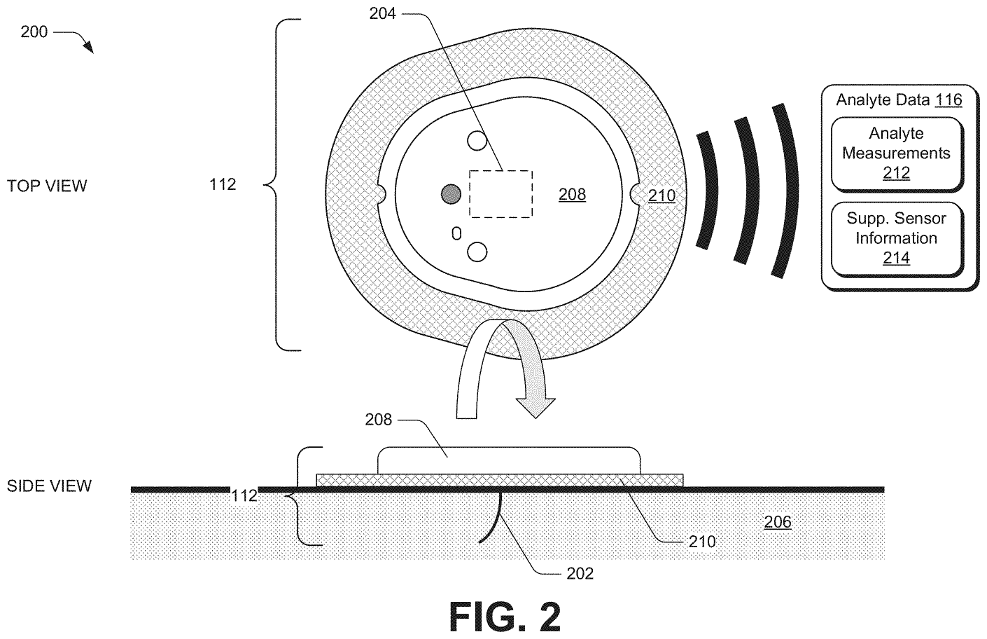

In one or more implementations, the wearable analyte monitoring device 112 is a continuous glucose monitoring (“CGM”) system. As used herein, the term “continuous” when used in connection with analyte monitoring may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when the computing device 106 establishes a wireless connection with the augmented analyte monitoring system 104 to retrieve one or more of the measurements), and so forth. This functionality along with further aspects of the configuration of the wearable analyte monitoring device 112 are discussed in more detail in relation to FIG. 2.

The analyte augmentation wearable 114 is configured to provide information related to the person 102 that is different from the analyte for which the wearable analyte monitoring device 112 is deployed, such as measurements of different analytes, measurements of various detected signals (e.g., biopotential measurements of the person 102 such as ECG, EMG, and/or EEG; acceleration experienced by the person 102 at a location where the analyte augmentation wearable 114 is worn), measurements of various physiological conditions (e.g., perspiration), or indications of detected events (e.g., exceeding or falling below a threshold, detecting the presence or absence of a particular compound), to name just a few. For example, the analyte augmentation wearable 114 may be configured with an architecture to sense one or more biochemical analytes in the person 102's sweat (e.g., through an adhesive patch) and generate perspiration measurements. Examples of analytes associated with perspiration include urea, uric acid, ionic potassium, ionic sodium, ionic chloride, etc. Alternatively, the analyte augmentation wearable 114 may be configured with electrodes configured to contact the person 102's skin and detect biopotential changes on the skin or transcutaneously, e.g., that result from the person 102's heart as it beats. The analyte augmentation wearable 114 may be configured with a variety of sensors without departing from the spirit or scope of the described techniques to provide additional physiological data about the person 102, such as temperature sensors, accelerometers, ultrasonic sensors, strain sensors, and additional analyte sensors, to name just a few.

In addition, the analyte augmentation wearable 114 may be configured with one or more sensors and/or an architecture to produce and detect light-based phenomena and generate various photonic measurements. In one or more implementations, configuration of the analyte augmentation wearable 114 with an architecture for producing and/or detecting photonic events, can enable the wearable to produce additional physiological data 118 that includes a variety of measurements, such as one or more of: heart rate of the person 102, heart rate variability of the person 102, partial pressure of oxygen (pO2) of the person 102, saturation of oxygen in the muscles (smO2) of the person 102, oxygen saturation (SpO₂) of the person 102, blood pressure of the person 102, and/or respiration rate of the person 102, to name just a few.

Alternatively or additionally, the analyte augmentation wearable 114 can include biopotential electrodes produce additional physiological data 118 corresponding to one or more of an electrocardiogram (EKG) for the person 102, electromyography (EMG) of the person 102, or an electroencephalogram (EEG) for the person 102. In one or more implementations where the analyte augmentation wearable 114 has an architecture that configures it as a biopotential monitoring device, the signals detected by the analyte augmentation wearable 114 can be used in combination with a separate wearable biopotential monitoring device (e.g., an EKG on a smart watch) to add “leads” (i.e., more sensors at different locations on the person 102's body) to increase a fidelity of an EKG that combines the signals detected using the multiple devices.

The described systems can also use biopotential electrodes to produce additional physiological data 118 describing changes in blood pressure using pulse transit time and for detecting seizures. The described systems can use an accelerometer to produce additional physiological data 118 describing activities (i.e., for “activity tracking”), faults (i.e., for fault detection), gait disturbances, and central tremors, to name just a few. The described systems can use temperature sensors to produce additional physiological data 118 describing temperature compensation, fever, ovulation, fault detection, temperature patterns assessment, and so forth. The described systems can use ultrasonic sensors to produce additional physiological data describing blood pressure of the person 102. The described systems can use strain sensors to produce additional physiological data 118 describing gait disturbances, central tremors, and recognized human activities, for instance. The information provided by such sensors may correspond to or otherwise be packaged for communication to the computing device 106 as additional physiological data 118.

By selecting one or more of a variety of available analyte augmentation wearables, for deployment with the wearable analyte monitoring device 112, the augmented analyte monitoring system 104 can be easily customized, e.g., by “mixing and matching” which analyte augmentation wearable is deployed with the wearable analyte monitoring device 112. In this way, different combinations of the analyte augmentation wearable 114 and the wearable analyte monitoring device 112 can be used to augment the analyte data 116 in numerous ways. Moreover, by mixing and matching which analyte augmentation wearable is deployed with the wearable analyte monitoring device 112, the augmented analyte monitoring system 104 can be customized, for instance, for different populations of patients and/or different types of health conditions. This enables the customized system to target analytes and other physiological signals of interest for the different populations and/or health conditions.

In one or more implementations, the augmented analyte monitoring system 104 transmits the analyte data 116 and the additional physiological data 118 as an augmented analyte packet 120 to the computing device 106, such as via a wireless connection for handling by a sensor hub 122 of the computing device 106. The augmented analyte monitoring system 104 may communicate the data in real-time, e.g., as it is produced using an analyte sensor or other architecture. Alternatively or in addition, the augmented analyte monitoring system 104 may communicate the data to the computing device 106 at predefined intervals of time. For example, the augmented analyte monitoring system 104 may be configured to communicate the augmented analyte packets 120 to the computing device 106 approximately every five minutes (as they are being produced).

Certainly, an interval at which the analyte data 116 and the additional physiological data 118 are communicated may be different from the examples above without departing from the spirit or scope of the described techniques. The data may be communicated by the augmented analyte monitoring system 104 to the computing device 106 according to other bases in accordance with the described techniques, such as based on a request from the sensor hub 122. Regardless, the computing device 106 may maintain the analyte data 116 and the additional physiological data 118 at least temporarily, e.g., in a storage device 124 of the computing device 106. The analyte data 116 and the additional physiological data 118 may also be maintained in the storage device 124 with other associated data, such as corresponding timestamps and/or identifiers of respective augmented analyte packets 120 in which communicated, to name just a few.

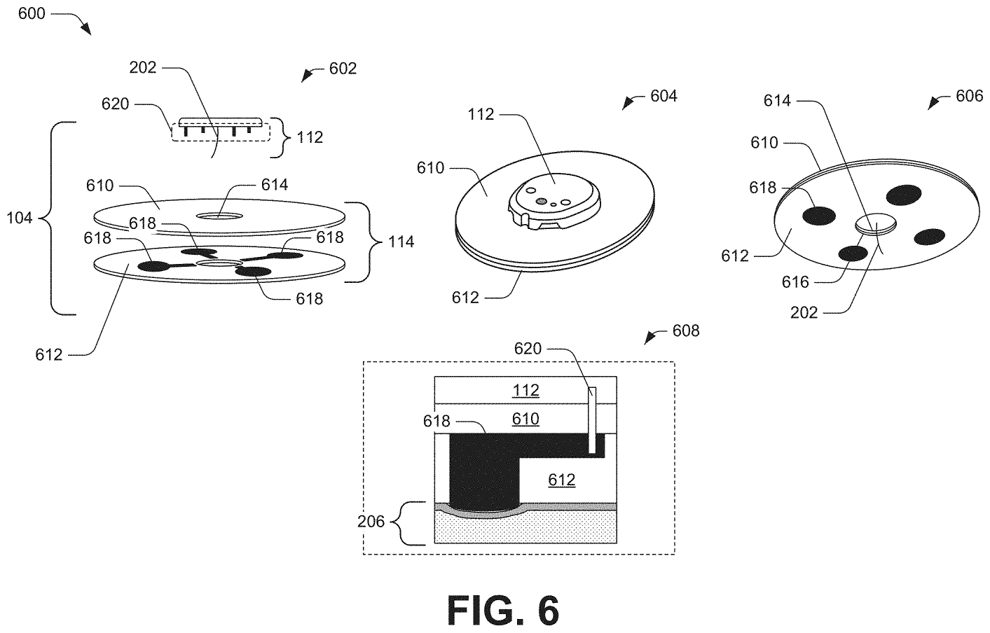

The wearable analyte monitoring device 112 and the analyte augmentation wearable 114 may be configured and combined in a variety of ways to form the augmented analyte monitoring system 104. By way of example, the analyte augmentation wearable 114 may be configured as an underlay patch with one or more sensors, e.g., to produce the additional physiological data 118. In configurations as an underlay patch, the analyte augmentation wearable 114 may be configured to be disposed at least partially between the wearable analyte monitoring device 112 and the skin of the person 102 when deployed. This position of the analyte augmentation wearable 114 as an underlay patch, between the wearable analyte monitoring device 112 and the skin of the person 102, may be referred to as “under” the wearable analyte monitoring device 112. In one example of this configuration, the analyte augmentation wearable 114 may be applied to the person 102's skin, and then the wearable analyte monitoring device 112 may be applied “on top” of the already applied analyte augmentation wearable 114. An example of the analyte augmentation wearable 114 as an underlay patch is discussed in more detail in relation to FIG. 6.

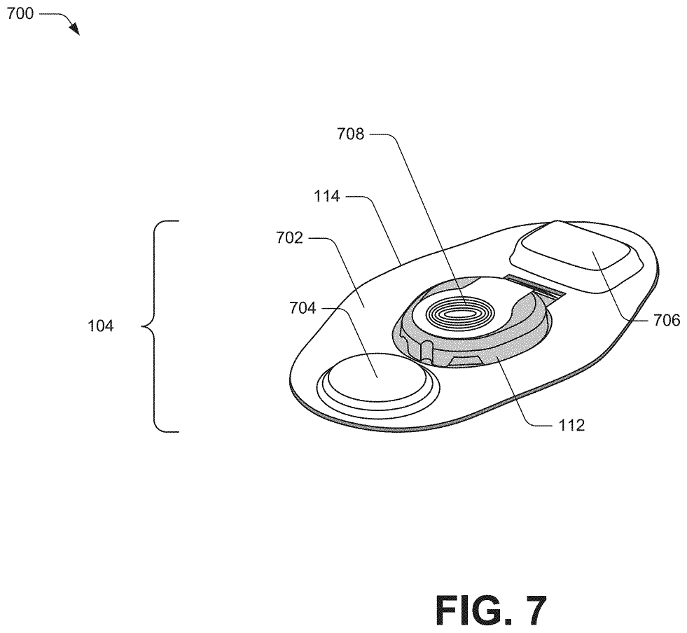

In one or more implementations, the analyte augmentation wearable 114 may be configured as an overlay patch with one or more sensors, e.g., to produce the additional physiological data 118. In configurations as an overlay patch, the analyte augmentation wearable 114 may be configured to be disposed at least partially covering the wearable analyte monitoring device 112, such that when deployed the wearable analyte monitoring device 112 is disposed at least partially between the analyte augmentation wearable 114 and the skin of the person 102. This position of the analyte augmentation wearable 114 as an overlay patch, on top of or at least partially covering the wearable analyte monitoring device 112, may be referred to as “over” the wearable analyte monitoring device 112. In one example of this configuration, the wearable analyte monitoring device 112 may be applied to the person 102's skin, and then the analyte augmentation wearable 114 may be applied “on top” of the already applied wearable analyte monitoring device 112. An example of the analyte augmentation wearable 114 as an underlay patch is discussed in more detail in relation to FIG. 7.

Broadly speaking, the analyte augmentation wearable 114 has a form factor that is complementary with a form factor of the wearable analyte monitoring device 112. In one or more implementations, for instance, the wearable analyte monitoring device 112 and the analyte augmentation wearable 114 are separate physical items that may be combined when they are applied one at a time to the person 102, such as when the analyte augmentation wearable 114 is applied (e.g., adhered) to the person 102's skin (e.g., as an underlay) and the wearable analyte monitoring device 112 is applied “on top” of the applied analyte augmentation wearable 114. Alternatively, the wearable analyte monitoring device 112 and the analyte augmentation wearable 114 may be separate physical items that are combined together (e.g., by the person 102 or a health care provider) before the combination is applied together to the person 102's skin.

At least one advantage of configuring the analyte augmentation wearable 114 as a separate form factor from the wearable analyte monitoring device 112 is that a user (e.g., the person 102, a health care provider, or other) can pick and choose different available analyte augmentation wearables to create a custom portfolio of sensing, and thus data production. This can be used to generate particular insights targeted to particular patient populations and/or health conditions. In one or more implementations, for instance, the wearable analyte monitoring device 112 may include multiple insertable (e.g., subcutaneously) sensors, such as to measure the person 102's glucose, lactate, ketones, uric acid, and so on. Here, one or more different analyte augmentation wearables, each capable of sensing different analytes and/or physiological signals, may be selected and deployed (e.g., as an overlay or underlay) for combination with the wearable analyte monitoring device 112. By supporting this mixing and matching, the augmented analyte monitoring system 104 and the sensor hub 122 produce data to describe a robust number of health conditions, and potentially enabling improved treatment and/or support in relation to those conditions.

Regardless, the form factor of the analyte augmentation wearable 114 may be complementary with the form factor of the wearable analyte monitoring device 112 such that the wearable analyte monitoring device 112 is not impeded by the analyte augmentation wearable 114 from producing the analyte data 116 in the same manner as if the wearable analyte monitoring device 112 were not combined with the analyte augmentation wearable 114. An analyte sensor of the wearable analyte monitoring device 112, for instance, may still be subcutaneously inserted into the skin of the person 102 to detect signals indicative of the analyte when used for the augmented analyte monitoring system 104.

Examples of features that cause a form factor of the analyte augmentation wearable 114 to be “complementary” with a form factor of the wearable analyte monitoring device 112 may include one or more of the following: an access (e.g., a cutout, hole, or membrane, to name a few) of the analyte augmentation wearable 114 that allows an analyte sensor of the wearable analyte monitoring device 112 to pass through the access and into the person 102's skin, an access (e.g., a cutout, geometry, hole, or membrane) of the analyte augmentation wearable 114 that fits around the wearable analyte monitoring device 112 (e.g., so that the analyte augmentation wearable 114 is configured to be applied to the person's skin around the wearable analyte monitoring device 112), a cavity having a complementary shape to the wearable analyte monitoring device 112 such that the wearable analyte monitoring device 112 fits within the cavity of the analyte augmentation wearable 114 and is covered when applied to the person's skin, a partial cavity having a complementary shape to the wearable analyte monitoring device 112 such that a portion of the wearable analyte monitoring device 112 fits within the partial cavity of the analyte augmentation wearable 114 and such that the portion of the wearable analyte monitoring device 112 is covered when applied while another portion of the wearable analyte monitoring device 112 is exposed (e.g., to air or the person 102's clothing), and so forth. It is to be appreciated that a form factor of the analyte augmentation wearable 114 may be complementary with a form factor of the wearable analyte monitoring device 112 in other ways without departing from the spirit or scope of the described techniques.

In one or more implementations, the analyte augmentation wearable 114 may not only have a complementary form factor with the wearable analyte monitoring device 112 but also one or more components that interface with (e.g., physically contact and/or communicably couple with) at least a portion of the wearable analyte monitoring device 112. By way of example, a patch portion of the analyte augmentation wearable 114 may surround the person 102's skin and a power/communication component (e.g., supporting wireless power, body area network, and/or near field communication (NFC)), may contact at least a portion of a housing of the wearable analyte monitoring device 112.

Additionally, the wearable analyte monitoring device 112 and the analyte augmentation wearable 114 may be communicably coupled in one or more implementations. By way of example, such a communicative coupling may enable the analyte augmentation wearable 114 to communicate signals and/or data that is received by the wearable analyte monitoring device 112. Alternatively, such a communicative coupling may enable the wearable analyte monitoring device 112 to communicate signals and/or data that is received by the analyte augmentation wearable 114. Alternatively, such a communicative coupling may enable two-way communication, such that the coupling enables both the wearable analyte monitoring device 112 and the analyte augmentation wearable 114 to communicate data to and receive data from the other wearable.

A communicative coupling between the wearable analyte monitoring device 112 and the analyte augmentation wearable 114 may be configured as a “wired” or “wireless” coupling. As used herein, a “wired” coupling refers to a physical connection of components capable of transferring data, e.g., the analyte data 116 and/or the additional physiological data 118, from one device to another. In one or more implementations, the wearable analyte monitoring device 112 may include one or more of the following for establishing a “wired” communicative coupling with the analyte augmentation wearable 114: pins (e.g., that insert into ports of the analyte augmentation wearable 114 or penetrate sensors of the analyte augmentation wearable 114), ports capable of receiving pins of the analyte augmentation wearable 114, or contacts (e.g., to touch contacts or sensor components of the analyte augmentation wearable 114), to name just a few. To enable a “wired” coupling, the analyte augmentation wearable 114 may also be configured with one or more of pins, ports, and/or contacts, in one or more implementations. It is to be appreciated that the wearable analyte monitoring device 112 and the analyte augmentation wearable 114 may be configured in other ways to enable a wired connection between the two devices without departing from the spirit or scope of the techniques described herein.

As used herein, a “wireless” coupling refers to a coupling that involves transmission of a signal by one device (or component) along with detection and interpretation of the signal by a second device (or component), where at least a portion of the transmission, detection, and interpretation cross a span that is not hardwired. To enable a “wireless” communicative coupling with the wearable analyte monitoring device 112, for instance, the analyte augmentation wearable 114 may be configured with one or more wireless transmitters to transmit data (e.g., the additional physiological data 118). By way of example, the analyte augmentation wearable 114 may be configured to transmit data to the wearable analyte monitoring device 112 using one or more of NFC, Bluetooth, 5G, or a body area network, to name just a few. The analyte augmentation wearable 114 may be configured in various ways to transmit data wirelessly to the wearable analyte monitoring device 112 without departing from the described techniques, such as using changes in electric potential over skin of the person 102's body and using light (e.g., causing an LED to emit light at one or more known frequencies onto the person 102's skin and/or in a direction of a light detection component of the wearable analyte monitoring device 112).

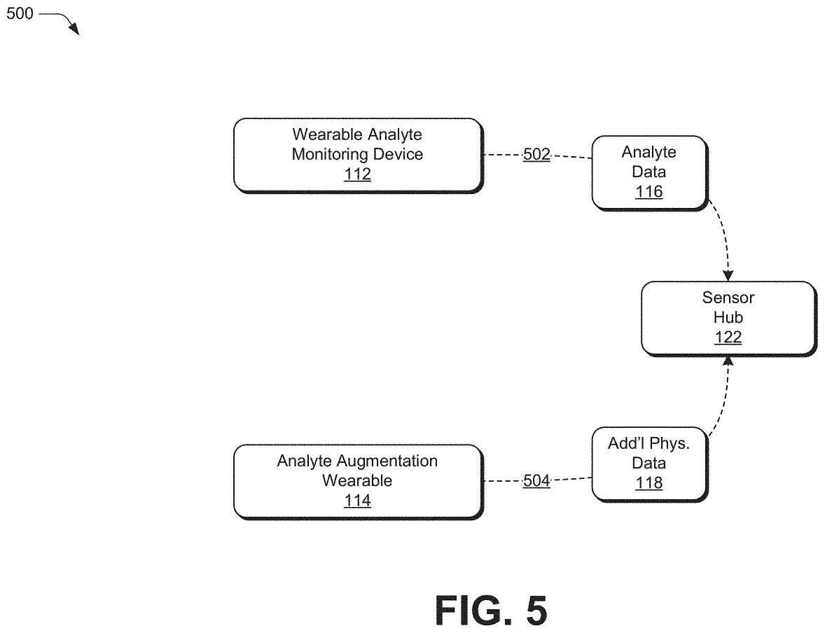

In scenarios where the wearable analyte monitoring device 112 communicates the analyte data 116 to the analyte augmentation wearable 114, the wearable analyte monitoring device 112 may be configured with one or more wireless transmitters to transmit data (e.g., the analyte data 116). By way of example, the wearable analyte monitoring device 112 may be configured to transmit data to the analyte augmentation wearable 114 using one or more of NFC, Bluetooth (BLE), or 5G, to name just a few. The wearable analyte monitoring device 112 may be configured in other ways to transmit data wirelessly to the analyte augmentation wearable 114 without departing from the described techniques, such as over skin of the person 102's body or using light. As discussed in more detail in relation to FIG. 5, both the wearable analyte monitoring device 112 and the analyte augmentation wearable 114 may be configured to establish a wireless connection with the sensor hub 122 and communicate data (e.g., the analyte data 116 and the additional physiological data 118 respectively) wirelessly over the established connection to the sensor hub 122.

As noted above, the sensor hub 122 may be implemented at the computing device 106, in one or more implementations. The computing device 106 may be configured in a variety of ways without departing from the spirit or scope of the described techniques. By way of example and not limitation, the computing device 106 may be configured as a mobile device (e.g., a mobile phone, a wearable device, or tablet device), a desktop computer, or a laptop computer, to name just a few form factors. In one or more implementations, the computing device 106 may be configured as a dedicated device associated with the health monitoring platform 108 and having the sensor hub 122. As a dedicated device associated with the health monitoring platform 108, the sensor hub 122 may be configured with functionality to obtain the analyte data 116 and the additional physiological data 118 from the augmented analyte monitoring system 104, perform various computations in relation to that data, display information related to the data and the health monitoring platform 108, communicate the data to the health monitoring platform 108, and so forth.

Additionally, the computing device 106 may be representative of more than one device in accordance with the described techniques. In one or more scenarios, for instance, the computing device 106 may correspond to both a wearable device (e.g., a smart watch, mouthguard, contact lenses, smart glasses, chest strap, ear buds, or headphones, to name just a few) and a mobile phone. In such scenarios, both of these devices may be capable of performing at least some of the same operations, such as to receive the analyte data 116 and the additional physiological data 118 from the augmented analyte monitoring system 104, communicate that data via the network 110 to the health monitoring platform 108, display information related to the data, and so forth. Alternatively or in addition, different devices may have different capabilities that other devices do not have or that are limited through computing instructions to specified devices. The computing device 106 and/or the wearable analyte monitoring device 112 may also be communicably coupled to one or more medical devices, in accordance with the described techniques, such as an insulin pump or implant. Due to this coupling, treatment may be administered using such medical devices based on determinations made by processing the analyte data 116 and the additional physiological data 118.

Turning now to a discussion of the sensor hub 122, the sensor hub 122 may be configured to receive the augmented analyte packet 120 from the augmented analyte monitoring system 104. In one or more implementations, the sensor hub 122 parses the augmented analyte packet 120 as received and augments the analyte data 116 by causing the analyte data 116 to be stored in association with the additional physiological data 118 in the storage device 124. In other words, the sensor hub 122 may modify the augmented analyte packet 120 for storage and/or extract the analyte data 116 and the additional physiological data 118 from the augmented analyte packet 120 and store the extracted data with associated data, such as by associating time stamps with the extracted data, performing computations on some of the data (e.g., computing statistics on some of the data and storing the computed statistics with the data), interpolating missing data, identifying erroneous data, and so forth. By way of example, the sensor hub 122 may build and/or populate a database in the storage device 124 with the data from the augmented analyte packet 120 or with data the sensor hub 122 derives from that data.

In one or more implementations, for instance, the sensor hub 122 may augment the analyte data 116 by modifying it with the additional physiological data 118. For example, in a scenario where the additional physiological data 118 corresponds to temperature data, the sensor hub 122 may compute temperature-corrected analyte measurements based on the analyte data 116 and the additional physiological data 118 and then cause those temperature-corrected analyte measurements to be stored in the storage device 124 in addition to or instead of the analyte data 116. This augmented analyte data may then be used in connection with one or more services provided to the user.

In the illustrated environment 100, the computing device 106 includes a health monitoring application 126. The health monitoring application 126 may provide one or more services by using the augmented analyte data. By way of example, the health monitoring application 126 may output the augmented analyte data, e.g., temperature-corrected analyte measurements, rather than output or in addition to outputting the analyte data 116 produced by the wearable analyte monitoring device 112. In one or more implementations, the health monitoring application 126, may output a trace of the augmented analyte data instead of or in addition to the analyte data 116 as produced by the wearable analyte monitoring device 112.

In accordance with the described techniques, the analyte data 116 may be augmented with the additional physiological data 118 to generate information and/or content that is more robust (e.g., accurate or actionable) than when the analyte data 116 is not augmented with the additional physiological data 118. In one or more implementations, the sensor hub 122, the health monitoring application 126, or the health monitoring platform 108 may generate such information that is more robust by using the combination of the analyte data 116 and the additional physiological data 118 rather than by simply using the analyte data 116. Examples of the information that may be generated using the augmented analyte data and which may be more accurate and/or actionable due to using the augmented analyte data include, for instance, reports, user interfaces that plot estimated values as received, and notifications of events or predicted events, to name just a few.

Alternatively or additionally, the analyte data 116 may be usable to confirm events captured by the additional physiological data 118, e.g., cardiac events. Alternatively or additionally, the analyte data 116 may be used to modify the additional physiological data 118 to make it more accurate, e.g., to confirm that a meal was eaten. The additional physiological data 118 may augment the analyte data 116 in a variety of ways to improve determinations made about the person 102's health in relation to determinations made using the analyte data 116 without the additional physiological data 118. For example, the additional physiological data 118 may be used to generate insights related to one or more conditions (e.g., diabetes, heart disease, etc.) for which the analyte data 116 may also be collected, and the additional physiological data 118 may be used to generate insights in relation to one or more conditions that are complementary to insights derived from the analyte data 116. Moreover, co-location of the analyte augmentation wearable 114's one or more sensors and one or more analyte sensors of the wearable analyte monitoring device 112 enables the data to be attributed to proximal locations on the person 102's body and correlated. This contrasts with measurements produced at different parts of the body. In the context of measuring the analyte, e.g., glucose continuously, and obtaining analyte data describing such measurements, consider the following discussion of FIG. 2.

FIG. 2 depicts an example 200 of an implementation of the wearable analyte monitoring device 112 in greater detail. In particular, the illustrated example 200 includes a top view and a corresponding side view of the wearable analyte monitoring device 112. It is to be appreciated that the wearable analyte monitoring device 112 may vary in implementation from the following discussion in various ways without departing from the spirit or scope of the described techniques.

In this example 200, the wearable analyte monitoring device 112 is illustrated to include an analyte sensor 202 (e.g., a glucose sensor) and a sensor module 204. Here, the analyte sensor 202 is depicted in the side view having been inserted subcutaneously into skin 206, e.g., of the person 102. The sensor module 204 is approximated in the top view as a dashed rectangle. The wearable analyte monitoring device 112 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the computing device 106, may also be housed or otherwise implemented within the housing of the transmitter 208. In this example 200, the wearable analyte monitoring device 112 further includes adhesive pad 210, e.g., for adhering the wearable analyte monitoring device 112 to the skin 206.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, user-initiated application of the wearable analyte monitoring device 112 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or healthcare provider.

The wearable analyte monitoring device 112 may also be removed by peeling the adhesive pad 210 from the skin 206. It is to be appreciated that the wearable analyte monitoring device 112 and its various components as illustrated are simply one example form factor, and the wearable analyte monitoring device 112 and its components may have different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively and these communications can be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase which reacts with glucose and oxygen to form hydrogen peroxide that is electrochemically detectable by the sensor module 204 which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve accuracy in identifying or predicting glucose-based events. Additionally or alternatively, the wearable analyte monitoring device 112 may include additional sensors and/or architectures to the analyte sensor 202 to detect those analytes indicative of the other markers.

In another example, the analyte sensor 202 (or an additional sensor of the wearable analyte monitoring device 112—not shown) can include a first and second electrical conductor and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively or additionally, the wearable analyte monitoring device 112 includes multiple sensors to detect not only one or more analytes (e.g., ionic sodium, ionic potassium, carbon dioxide, glucose, and insulin) but also one or more environmental conditions (e.g., temperature). Thus, the sensor module 204 and the analyte sensor 202 (as well as any additional sensors) may detect the presence of one or more analytes, the absence of one or more analytes, and/or changes in one or more environmental conditions. As noted above, the wearable analyte monitoring device 112 may be configured to produce data describing a single analyte (e.g., glucose) or multiple analytes. Further, a combination of the analytes for which wearable analyte monitoring devices are configured may vary across different lots of the monitoring devices manufactured (e.g., by the health monitoring platform 108), such that wearable analyte monitoring devices having different architectures may be configured for use by different patient populations and/or for different health conditions.

In one or more implementations, the sensor module 204 may include a processor and memory (not shown). The sensor module 204, by leveraging the processor, may generate analyte measurements 212 based on the communications with the analyte sensor 202 that are indicative of the above-discussed changes. Based on the above-noted communications from the analyte sensor 202, the sensor module 204 is further configured to generate communicable packages of data that include at least one analyte measurement 212. In this example 200, the analyte data 116 represents these packages of data. Additionally or alternatively, the sensor module 204 may configure the analyte data 116 to include additional data, including, by way of example, supplemental sensor information 214. The supplemental sensor information 214 may include a sensor identifier, a sensor status, temperatures that correspond to the analyte measurements 212, measurements of other analytes that correspond to the analyte measurements 212, and so forth. It is to be appreciated that supplemental sensor information 214 may include a variety of data that supplements at least one analyte measurement 212 without departing from the spirit or scope of the described techniques.

In implementations where the wearable analyte monitoring device 112 is configured for wireless transmission, the transmitter 208 may transmit the analyte data 116 as a stream of data to a computing device. Alternatively or additionally, the sensor module 204 may buffer the analyte measurements 212 and/or the supplemental sensor information 214 (e.g., in memory of the sensor module 204 and/or other physical computer-readable storage media of the wearable analyte monitoring device 112) and cause the transmitter 208 to transmit the buffered analyte data 116 later at various regular or irregular intervals, e.g., time intervals (approximately every second, approximately every thirty seconds, approximately every minute, approximately every five minutes, approximately every hour, and so on), storage intervals (when the buffered analyte measurements 212 and/or supplemental sensor information 214 reach a threshold amount of data or a number of measurements), and so forth.

Having considered an example of an environment and an example of a wearable analyte monitoring device, consider now a discussion of some examples of details of the techniques for an augmented analyte monitoring system in accordance with one or more implementations.

Augmented Analyte Monitoring System

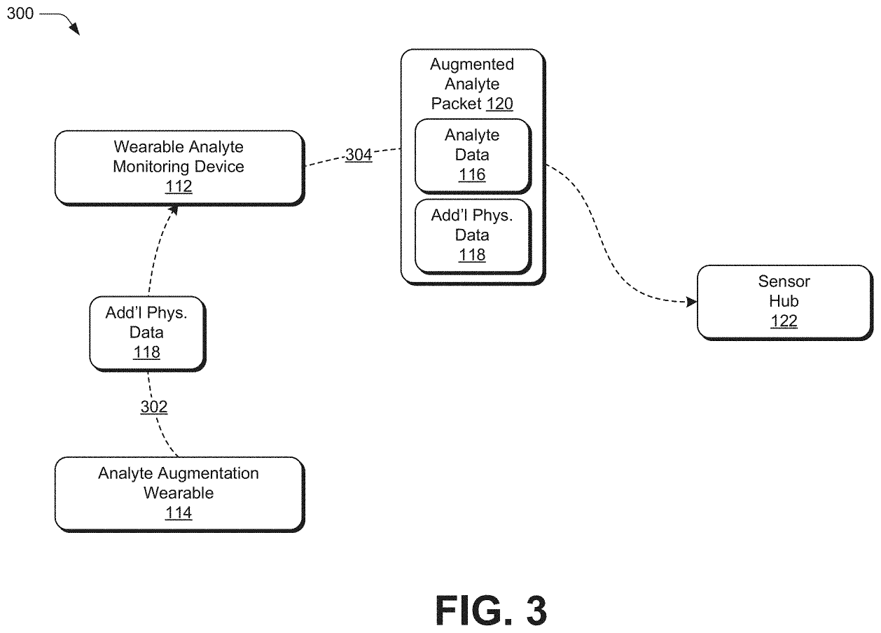

FIG. 3 depicts an example 300 of an implementation of augmenting analyte data from a wearable analyte monitoring device with additional physiological data from an analyte augmentation wearable. The illustrated example 300 includes from FIG. 1 the wearable analyte monitoring device 112, the analyte augmentation wearable 114, and the sensor hub 122.

In this example 300, the wearable analyte monitoring device 112 and the analyte augmentation wearable 114 are communicably coupled via coupling 302. Additionally, the wearable analyte monitoring device 112 and the sensor hub 122 are communicably coupled via coupling 304. In accordance with the described techniques, the coupling 302 between the wearable analyte monitoring device 112 and the analyte augmentation wearable 114 may be wired (or otherwise a physical coupling of signal transmitting and receiving components) or wireless (including using the body of the person 102 or light) for communicating and signals, examples of these types of couplings are discussed in more detail above. The coupling 304 between the wearable analyte monitoring device 112 and the sensor hub 122 may also be wired or wireless. One example scenario of a wired coupling between the wearable analyte monitoring device 112 and the sensor hub 122 may include connecting a cord between a computing device (e.g., the computing device 106) having the sensor hub 122 and the wearable analyte monitoring device 112 (or the augmented analyte monitoring system 104).

In this example 300, the analyte augmentation wearable 114 is depicted communicating the additional physiological data 118 over the coupling 302 to the wearable analyte monitoring device 112. Here, the wearable analyte monitoring device 112 may package the additional physiological data 118 obtained with the analyte data 116 (e.g., using the sensor module 204 and/or onboard processors) to form the augmented analyte packet 120. The wearable analyte monitoring device 112 may then transmit the augmented analyte packet 120 to the sensor hub 122 over the coupling 304, e.g., using transmitter 208. In one or more implementations, the analyte augmentation wearable 114 may use one or more compression techniques to compress the additional physiological data 118 for communication over the coupling 302 and/or the wearable analyte monitoring device 112 may use one or more compression techniques to compress the augmented analyte packet 120 for communication over the coupling 304.

The illustrated example 300 contrasts with implementations where the analyte augmentation wearable 114 communicates the augmented analyte packet 120 to the sensor hub 122 rather than the wearable analyte monitoring device 112. In the context of the analyte augmentation wearable 114 communicating the augmented analyte packet 120 to the sensor hub 122 rather than the wearable analyte monitoring device 112, consider the following discussion.

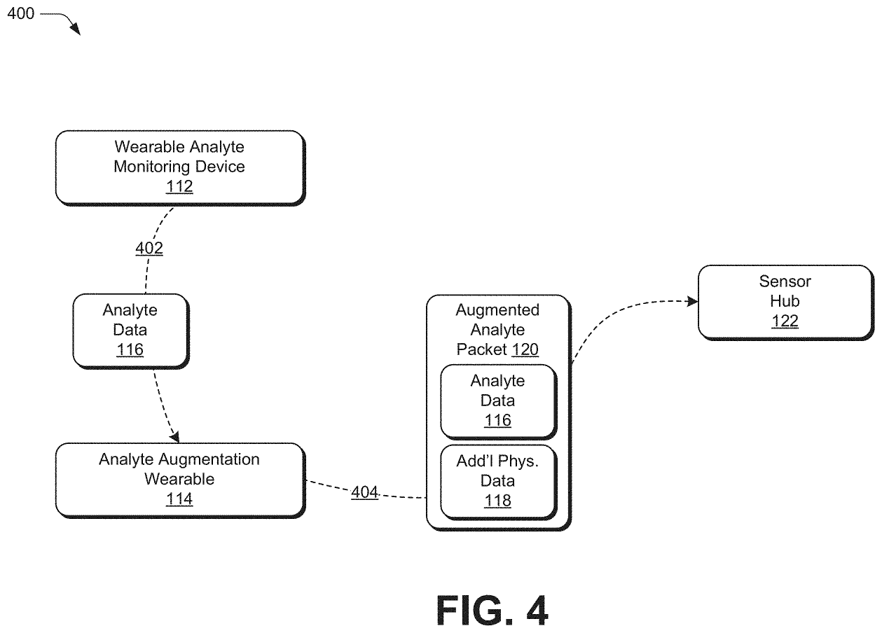

FIG. 4 depicts an example 400 of a first different implementation of augmenting analyte data from the wearable analyte monitoring device with additional physiological data from the analyte augmentation wearable. The illustrated example 400 includes from FIG. 1 the wearable analyte monitoring device 112, the analyte augmentation wearable 114, and the sensor hub 122.

In this example 400, the wearable analyte monitoring device 112 and the analyte augmentation wearable 114 are communicably coupled via coupling 402. Additionally, the analyte augmentation wearable 114 and the sensor hub 122 are communicably coupled via coupling 404. In accordance with the described techniques, the coupling 402 between the wearable analyte monitoring device 112 and the analyte augmentation wearable 114 may be wired (or otherwise a physical coupling of signal transmitting and receiving components) or wireless (including using the body of the person 102 or light) for communicating and signals, examples of these types of couplings are discussed in more detail above. The coupling 404 between the analyte augmentation wearable 114 and the sensor hub 122 may also be wired or wireless. One example scenario of a wired coupling between the wearable analyte augmentation wearable 114 and the sensor hub 122 may include connecting a cord between a computing device (e.g., the computing device 106) having the sensor hub 122 and the analyte augmentation wearable 114 (or the augmented analyte monitoring system 104).

In this example 400, the wearable analyte monitoring device 112 is depicted communicating the analyte data 116 over the coupling 402 to the analyte augmentation wearable 114. Here, the analyte augmentation wearable 114 may package the analyte data 116 obtained with the additional physiological data 118 (e.g., using onboard processors, computer-readable media, and/or other hardware components) to form the augmented analyte packet 120. The analyte augmentation wearable 114 may then transmit the augmented analyte packet 120 to the sensor hub 122 over the coupling 404. In one or more implementations, the wearable analyte monitoring device 112 may use one or more compression techniques to compress the analyte data 116 for communication over the coupling 402 and/or the analyte augmentation wearable 114 may use one or more compression techniques to compress the augmented analyte packet 120 for communication over the coupling 404.

FIG. 5 depicts an example 500 of a second different implementation of augmenting analyte data from the wearable analyte monitoring device with additional physiological data from the analyte augmentation wearable. The illustrated example 500 includes from FIG. 1 the wearable analyte monitoring device 112, the analyte augmentation wearable 114, and the sensor hub 122.

In this example 500, the wearable analyte monitoring device 112 and the sensor hub 122 are communicably coupled via coupling 502. Additionally, the analyte augmentation wearable 114 and the sensor hub 122 are communicably coupled via coupling 504. In accordance with the described techniques, the coupling 502 between the wearable analyte monitoring device 112 and the sensor hub 122 may be wired (or otherwise a physical coupling of signal transmitting and receiving components) or wireless, examples of these types of couplings are discussed in more detail above. Likewise, the coupling 504 between the analyte augmentation wearable 114 and the sensor hub 122 may be wired (or otherwise a physical coupling of signal transmitting and receiving components) or wireless, examples of these types of couplings are discussed in more detail above. One example scenario of a wired coupling between the wearable analyte monitoring device 112 and the sensor hub 122 may include connecting a cord between a computing device (e.g., the computing device 106) having the sensor hub 122 and wearable analyte monitoring device 112. An example scenario of a wired coupling between the wearable analyte augmentation wearable 114 and the sensor hub 122 may include connecting a cord between a computing device (e.g., the computing device 106) having the sensor hub 122 and the analyte augmentation wearable 114.

In this example 500, the wearable analyte monitoring device 112 is depicted communicating the analyte data 116 over the coupling 502 to the sensor hub 122, and the analyte augmentation wearable 114 is depicted communicating the additional physiological data 118 over the coupling 504 to the sensor hub 122. As noted above, the sensor hub 122 may process the analyte data 116 as augmented by the additional physiological data 118 in a variety of ways without departing from the spirit or scope of the techniques described herein, such as by deriving adjusted analyte values determined by adjusting the analyte data 116 based on the additional physiological data 118, by determining correspondences between the analyte data 116 and the additional physiological data 118, and/or by determining covariances in the signals described by the analyte data 116 and the additional physiological data 118 for populating a database in the storage device 124.

FIG. 6 depicts an example 600 of an implementation of an analyte augmentation wearable configured as an underlay to augment the wearable analyte monitoring device.

The illustrated example 600 includes from FIG. 1 the augmented analyte monitoring system 104, which includes the wearable analyte monitoring device 112 and the analyte augmentation wearable 114. In particular, the illustrated example 600 includes a plurality of views 602-608 of the augmented analyte monitoring system 104, including exploded view 602, top assembled view 604, bottom assembled view 606, and cross-sectional view 608.

The exploded view 602 depicts the wearable analyte monitoring device 112 and the analyte augmentation wearable 114, which in this example includes membrane 610 and underlay patch 612. In one or more implementations, the underlay patch 612 may be configured to be applied so that it directly contacts the skin 206 of the person 102. Additionally, the membrane 610 is configured to be applied or otherwise disposed against a face of the underlay patch 612 opposite the face that contacts the skin 206 when deployed. In one or more underlay configurations, as depicted here, a housing of the wearable analyte monitoring device 112 may generally be disposed on top of the membrane 610. Although a portion (e.g., a majority) of the wearable analyte monitoring device 112 may be disposed on the membrane 610 in such underlay configurations, the membrane 610 may include a membrane access 614 (e.g., a hole, cutout, puncturable portion) and the underlay patch 612 may include a corresponding patch access 616 (e.g., a hole, cutout, puncturable portion) that can be aligned when deployed. The alignment of these access portions enables the analyte sensor 202 of the wearable analyte monitoring device 112 to extend through those access portions and insert subcutaneously into the skin 206 of the person 102. These access portions may be cutout from the layers as specialized design elements to allow the analyte sensor 202 to pass through the layers and operate normally. The bottom assembled view 606 depicts the analyte sensor 202 extending through the membrane access 614 and the corresponding patch access 616.

As noted above, the analyte augmentation wearable 114 may be configured with one or more sensors used to detect changes in one or more conditions and to produce the additional physiological data 118 based on detected changes. For example, the underlay patch 612 may include one or more sensors. Here, for instance, the underlay patch 612 is depicted with a plurality of electrodes 618. In one or more implementations involving electrodes, the electrodes 618 may be used to detect biopotential changes on the skin of a person or transcutaneously, such as those due to the person's beating heart or due to brain activity. It is to be appreciated that an underlay patch may be configured with one or more additional or different sensors without departing from the spirit or scope of the techniques described herein, such as various electrochemical sensors (e.g., sweat sensors), optical sensors (e.g., PPG), or accelerometers, to name just a few. Alternatively or in addition, the analyte augmentation wearable 114 may include some combination of non-invasive, transdermal, and/or subcutaneous sensors.

In the illustrated example 600, the wearable analyte monitoring device 112 is depicted including pins 620. In one or more implementations, the pins 620 may be configured to contact sensors or communicable contacts of the analyte augmentation wearable 114 and produce the additional physiological data 118. As depicted, for instance, the pins 620 may contact the electrodes 618 to detect the electrical changes and to generate the additional physiological data 118. In the cross-sectional view 608, the pins 620 are depicted originating from the wearable analyte monitoring device 112, passing through the membrane 610 (e.g., puncturing it), and terminating in the electrodes 618 (e.g., puncturing the electrodes 618 also). With the pins 620 coupled to the sensors of the analyte augmentation wearable 114 (e.g., contacting the electrodes 618), the wearable analyte monitoring device 112 may detect condition changes (e.g., electrical changes) to produce the additional physiological data 118 and augment the analyte data 116 produced using the analyte sensor 202. Thus, in one or more implementations, the wearable analyte monitoring device 112 may be coupled with one or more portions of the analyte augmentation wearable 114 to produce the additional physiological data 118.

In the bottom assembled view 606 and the cross-sectional view 608, the electrodes 618 are depicted extending through the underlay patch 612. By extending through the underlay patch 612, the electrodes 618 may be disposed against the membrane 610 and also exposed on an opposite side so that they can physically contact the skin 206 when deployed. Although not depicted, one or more of the membrane 610 or the underlay patch 612 may include markings configured for aligning an applicator of the wearable analyte monitoring device 112. By manipulating an applicator so that it aligns with such markers, the markers enable users to more easily apply the wearable analyte monitoring device 112 so that the analyte sensor 202 is deployed through the membrane access 614 and the corresponding patch access 616.

With regard to the electrodes 618, incorporating biopotential electrodes, including gel electrolyte electrodes, into the analyte augmentation wearable 114 enables electrocardiograms (EKG) and/or heart rate recordings to be produced as the additional physiological data 118. When the additional physiological data 118 includes EKGs and/or heart rate recordings, the sensor hub 122 and/or the health monitoring application 126 can identify hypoglycemic events (e.g., hyper- and hypo-glycemia) based on patterns in the analyte data 116 and confirm the occurrence or non-occurrence of those events based on the additional physiological data 118. Alternatively or in addition, the electrodes 618 may be used for electromyography (EMG), a diagnostic procedure that evaluates the health condition of muscles and the cells that control them. It is to be appreciated that configurations of the analyte augmentation wearable 114 as an underlay apparatus may vary from the configurations described herein in accordance with the described techniques.

FIG. 7 depicts an example 700 of an implementation of an analyte augmentation wearable configured as an overlay to augment the wearable analyte monitoring device.

The illustrated example 700 includes from FIG. 1 the augmented analyte monitoring system 104, which includes the wearable analyte monitoring device 112 and the analyte augmentation wearable 114. In the illustrated example 700, the analyte augmentation wearable 114 is configured as an overlay patch. As noted above, in overlay configurations, the analyte augmentation wearable 114 is configured to be applied on top of the wearable analyte monitoring device 112, such as when the wearable analyte monitoring device 112 has already been deployed. By way of example, the analyte augmentation wearable 114 may have adhesive for adhering the analyte augmentation wearable 114 to skin and the wearable analyte monitoring device 112 after the wearable analyte monitoring device 112 is deployed.

In this example 700, the analyte augmentation wearable 114 configured as an overlay patch includes a patch portion 702, a first housing 704, and a second housing 706. The patch portion 702 may include adhesive on a face of the patch portion 702 that is configured to contact the skin 206 of the person 102. In the illustrated example 700, this face is occluded from view by an outer face of the patch portion 702. In one or more implementations, the adhesive is configured to apply the analyte augmentation wearable 114 to the skin 206 of the person 102 and on top of the wearable analyte monitoring device 112. The adhesive is generally configured to hold the analyte augmentation wearable 114, where deployed on the person 102, for a period of time, e.g., a period of wear of the analyte augmentation wearable 114. The adhesive may also be configured to allow a person to remove the analyte augmentation wearable 114 without injury generally.

The first housing 704 may be configured to house one or more of: sensors of the analyte augmentation wearable 114, a power source for the analyte augmentation wearable 114 and/or the wearable analyte monitoring device 112, a transmitter, and/or a receiver, to name just a few. Similarly, the second housing 706 may be configured to house one or more of: sensors of the analyte augmentation wearable 114, a power source for the analyte augmentation wearable 114 and/or the wearable analyte monitoring device 112, a transmitter, and/or a receiver, to name just a few. In one or more implementations, the patch portion 702 may also include one or more integrated sensors. In one or more implementations, the patch portion 702, the first housing 704, and/or the second housing 706 may include or otherwise incorporate one or more biopotential electrodes which are configured as one or more reference electrodes for the wearable analyte monitoring device 112, e.g., that enable the wearable analyte monitoring device 112 to produce measurements of the analyte using the electrodes.

Although not depicted, the patch portion 702 may include couplings to couple one or more of sensors, a power source, a receiver, and/or a transmitter. These couplings may couple such components for communication or supplying power. By way of example, the couplings may couple components housed in the first housing 704 with components housed the second housing 706 and/or with the wearable analyte monitoring device 112. The couplings may also couple components housed in the second housing 706 with components housed in the first housing 704 and/or with the wearable analyte monitoring device 112. The components may also couple components disposed throughout the patch portion 702 (e.g., sensors), one to another, and/or with components disposed at other portions of the analyte augmentation wearable 114 and/or with the wearable analyte monitoring device 112.

In one or more implementations, for instance, couplings in the patch portion 702 may couple sensors of the analyte augmentation wearable 114 to a transmitter to transmit data produced using the sensors (e.g., the additional physiological data 118), such as to communicate the data off the analyte augmentation wearable 114 to the wearable analyte monitoring device 112 and/or to the sensor hub 122. Alternatively or in addition, such couplings may couple a power source (e.g., a battery) to sensors to supply power that enables the sensors to operate. Alternatively or in addition, those couplings may couple a power source to a transmitter and/or a receiver to supply power that enables the transmitter and/or the receiver to operate, e.g., to transmit or receive data, respectively. It should be appreciated that while a separate transmitter and receiver may be discussed herein, in one or more implementations, a component may be configured to operate dually as a transmitter and a receiver.

Regarding powering the wearable analyte monitoring device 112 and/or the analyte augmentation wearable 114, in one or more implementations, the analyte augmentation wearable 114 may include a light sensitive material (e.g., on the patch portion 702). The light sensitive material of the analyte augmentation wearable 114 may be used to recharge a battery with light. An enlarged surface area, relative to a surface area of the wearable analyte monitoring device 112, may enable a sufficient amount of light to be used in order to charge a battery, which contrasts with a surface area of the wearable analyte monitoring device 112, which may not be large enough to sufficiently recharge a battery.

The patch portion 702, the first housing 704, and/or the second housing 706 may also include one or more processors and/or computer readable media, in one or more implementations. The above-discussed couplings may couple these components, one to another (e.g., similar to a bus), and/or to other components such as a power source and/or transmitter/receiver. The inclusion of processors and/or computer readable media may enable the analyte augmentation wearable 114 to process changes detected by sensors of the analyte augmentation wearable 114 and produce the additional physiological data 118 based on the detected changes. Such computer-readable media may also be configured to store, at least temporarily, data such as the additional physiological data 118 or the analyte data 116, e.g., before the data is communicated off the analyte augmentation wearable 114. It is to be appreciated, though, that in one or more implementations a receiver of the analyte augmentation wearable 114 may simply receive the analyte data 116 from the wearable analyte monitoring device 112 and communicate the data via an integrated transmitter, e.g., to the sensor hub 122.

In this context, the illustrated analyte augmentation wearable 114 includes coil 708, e.g., an NFC coil. It should be appreciated that the coil 708 may be used to implement different communication protocols, such as Bluetooth (BLE) or 5G, to name a couple. Regardless of a particular protocol, the coil 708 may be configured to receive the analyte data 116 transmitted by the wearable analyte monitoring device 112. The analyte augmentation wearable 114 may then route the received analyte data 116 to a transmitter, e.g., housed in the first housing 704 or the second housing 706. This transmitter of the analyte augmentation wearable 114 may be further configured to transmit the analyte data 116 along with the additional physiological data 118 produced by sensors of the analyte augmentation wearable 114. For example, such a transmitter may transmit this data to the sensor hub 122. This routing of data is discussed in more detail in relation to FIG. 4. Alternatively or in addition, the coil 708 may be configured to wirelessly transmit data (e.g., the additional physiological data 118) to the wearable analyte monitoring device 112. The wearable analyte monitoring device 112 may then transmit the additional physiological data 118 to the sensor hub, which is discussed in more detail in relation to FIG. 3.

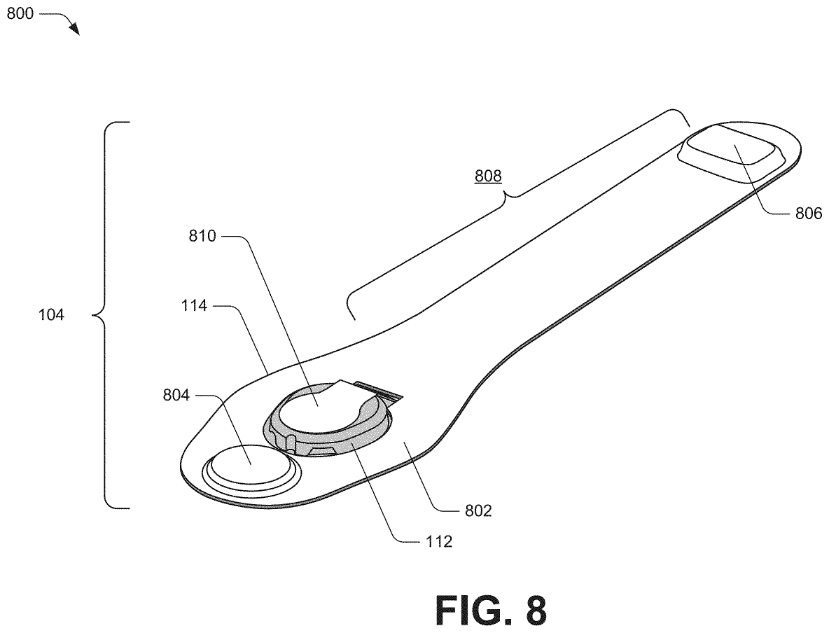

In contrast to the example overlay patch discussed in more detail in relation to FIG. 8, the patch portion 702 of the illustrated example 700 does not include a satellite extension. Accordingly, both the first housing 704 and the second housing 706 are located proximal the wearable analyte monitoring device 112. Due to this, sensors of the analyte augmentation wearable 114 are proximal to the analyte sensor 202 of the wearable analyte monitoring device 112. The analyte augmentation wearable 114's sensors thus are generally co-located to the wearable analyte monitoring device 112's analyte sensor (or its other sensors) when the analyte augmentation wearable 114 is configured similar to the depicted example. In other words, the patch portion 702 causes the analyte augmentation wearable 114's sensors to be deployed within a threshold distance of the wearable analyte monitoring device 112, where the position within the threshold distance corresponds to co-location of the sensors. It is to be appreciated that configurations of the analyte augmentation wearable 114 as an overlay apparatus may vary from the configurations described herein in accordance with the described techniques.

FIG. 8 depicts an example 800 of an implementation of an analyte augmentation wearable configured as an overlay with a satellite extension to augment the wearable analyte monitoring device.

The illustrated example 800 includes from FIG. 1 the augmented analyte monitoring system 104, which includes the wearable analyte monitoring device 112 and the analyte augmentation wearable 114. In the illustrated example 800, the analyte augmentation wearable 114 is configured as an overlay patch with a satellite extension. As noted above, in overlay configurations, the analyte augmentation wearable 114 is configured to be applied on top of the wearable analyte monitoring device 112, such as when the wearable analyte monitoring device 112 has already been deployed. Similar to the overlay patch discussion in FIG. 7, the analyte augmentation wearable 114 may have adhesive for adhering the analyte augmentation wearable 114 to skin and to the wearable analyte monitoring device 112 after the wearable analyte monitoring device 112 is deployed.

In this example 800, the analyte augmentation wearable 114 includes a patch portion 802, a first housing 804, a second housing 806, and satellite extension 808. The patch portion 802 may include adhesive on a face of the patch portion 802 that is configured to contact the skin 206 of the person 102. In the illustrated example 700, this face is occluded from view by an outer face of the patch portion 702.

The first housing 804 and the second housing 806 may be configured in a similar manner to the first housing 704 and the second housing 706, as discussed in relation to FIG. 7. For example, the first housing 804 and the second housing 806 may be configured to house one or more of: sensors, a power source, a transmitter, and/or a receiver, to name just a few. In accordance with the described techniques, the patch portion 802 may include one or more integrated sensors. In one or more implementations, the patch portion 802, the first housing 804, the second housing 806, and/or the satellite extension 808 may include or otherwise incorporate one or more biopotential electrodes which are configured as one or more reference electrodes for the wearable analyte monitoring device 112.

In addition, the patch portion 802 may include couplings to couple one or more of: sensors, a power source, a receiver, a transmitter, and/or the wearable analyte monitoring device 112. These couplings may couple such components for communication or supplying power. By way of example, the couplings may couple components housed in the first housing 804 with components housed in the second housing 806 and/or with the wearable analyte monitoring device 112. The couplings may also couple components housed in the second housing 806 with components housed in the first housing 804 and/or with the wearable analyte monitoring device 112. Some further examples of how couplings may be deployed in the patch portion 802 are discussed in relation to FIG. 7.

In one or more implementations where the analyte augmentation wearable 114 is configured as an overlay patch with a satellite extension, the analyte augmentation wearable 114 may also include one or more processors and/or computer readable media. Some examples of the functionality enabled by including these components in one or more of the patch portion 802, the first housing 804, the second housing 806, or the satellite extension 808, are discussed in relation to FIG. 7. The illustrated example 800 also depicts a coil 810. The coil 810 may be coupled to the patch portion 802 and, like in the example 700, may be used to implement different communication protocols, such as NFC, Bluetooth (BLE), or 5G, to name just a few. The coil 810 may be configured to transmit and/or receive data in similar manners as discussed in relation to the coil 708.

In contrast to the overlay patch discussed in relation to FIG. 7, the patch portion 802 of the illustrated example 800 includes the satellite extension 808. In general, the satellite extension 808 is configured to position one or more sensors or components of the analyte augmentation wearable 114 at least a threshold distance away from the wearable analyte monitoring device 112. In contrast to the patch portion 702, which is configured to generally co-locate sensors and/or components of the analyte augmentation wearable 114 with the wearable analyte monitoring device 112, the patch portion 802 is configured to position at least one sensor and/or component of the analyte augmentation wearable 114 at a remote (i.e., “satellite”) location of the augmented analyte monitoring system 104, where remote is relative to the wearable analyte monitoring device 112 or at least one other portion of the analyte augmentation wearable 114.

As used herein, a “remote” location refers to a location that is at least a threshold distance (a known distance based on a length of the satellite extension 808) from the wearable analyte monitoring device 112 or from a particular portion of the analyte augmentation wearable 114. In one or more implementations, the “threshold” distance may correspond to a distance that enables one or more components disposed at the remote location to operate without interfering with operation of the wearable analyte monitoring device 112 (or non-remotely positioned components of the analyte augmentation wearable 114) or to operate without being interfered with due to operation of the wearable analyte monitoring device 112 (or non-remotely positioned components of the analyte augmentation wearable 114). By configuring the patch portion 802 with the satellite extension 808, the patch portion 802 may control a position of one or more components (e.g., sensors) of the analyte augmentation wearable 114 relative to one another and relative to the analyte sensor 202 of the wearable analyte monitoring device 112 (or relative to one or more other sensors or components of the wearable analyte monitoring device 112). Additionally, the satellite extension 808 enables the analyte augmentation wearable 114 to be deployed so that portions of the analyte augmentation wearable 114 at ends of the satellite extension 808 can be positioned over particular body parts or known distances from the wearable analyte monitoring device 112.

For example, the satellite extension 808 may enable the wearable analyte monitoring device 112 to be deployed at an abdomen of a person and the second housing 806 to be deployed concurrently at a lower back of the person. As another example, the satellite extension 808 may enable the wearable analyte monitoring device 112 to be deployed at a lateral portion of a person's arm and the second housing 806 to be deployed concurrent at a medial portion of the person's arm, such that the satellite extension 808 extends across the person's biceps or triceps. Certainly, a length of the satellite extension 808 may control how far away the satellite portion of the analyte augmentation wearable 114 is positioned away from other portions of the analyte augmentation wearable 114 and/or the wearable analyte monitoring device 112. Moreover, the satellite extension 808 may enable positioning of the portions relative to different body parts than those discussed just above without departing from the spirit or scope of the described techniques. It is also to be appreciated that configurations of the analyte augmentation wearable 114 as an overlay apparatus with a satellite extension may vary from the configurations described herein in accordance with the described techniques.

In one or more implementations, deployment of the analyte augmentation wearable 114, such that the satellite extension 808 positions the second housing 806 remotely from the first housing 804 and/or the wearable analyte monitoring device 112, enables metrics and/or various physiological data to be produced that is based on distance between sensors. This is because a distance between the second housing 806 and the first housing 804 and/or the wearable analyte monitoring device 112 are known. An example of these metrics and/or physiological data include at least pulse transit time (PTT), which is based on distance between sensors. Due to the known distance, the analyte augmentation wearable 114 can also be configured to produce multi-lead ECG measurements, such as by using electrodes incorporated with the overlay patch configuration (or underlay when configured as an underlay with a satellite), e.g., incorporated with the sensor-adjacent portion and incorporated with the portion across the satellite extension 808. Alternatively or additionally, the analyte augmentation wearable 114 can be configured for multi-lead ECG measurements using one or more electrodes of the sensor-adjacent portion of the patch, one or more electrodes of the portion across the satellite extension 808, and/or one or more electrodes of another, separate device (e.g., a smart watch). It is to be appreciated that various other metrics and/or physiological data may be produced based on a known distance by utilizing the described system and without departing from the spirit or scope of the described techniques.

With inclusion of the satellite extension 808, the analyte augmentation wearable 114 may have more surface area than configurations of the analyte augmentation wearable 114 discussed in relation to other examples. Due to this larger surface area, in one or more implementations, the satellite extension 808 may be utilized to recharge a battery (e.g., of the wearable analyte monitoring device 112 or the analyte augmentation wearable 114). By way of example, the satellite extension 808 may be manufactured at least in part to include light sensitive material to produce power from external background light. Alternatively or in addition, the satellite extension 808 may include an architecture that enables it to use sweat of the person 102's body as fuel, e.g., and thus charge a battery of the wearable analyte monitoring device 112 and/or the analyte augmentation wearable 114.

In the overlay configurations depicted in FIGS. 7 and 8 a form factor of the analyte augmentation wearable 114 is complementary to a form factor of the wearable analyte monitoring device 112. This is because a geometry of the patch portion 702 and the patch portion 802 (and the satellite extension 808) allow for deployment surrounding the wearable analyte monitoring device 112, e.g., there is a suitable access within which the wearable analyte monitoring device 112 may be disposed and at least partially surrounded by the overlay.

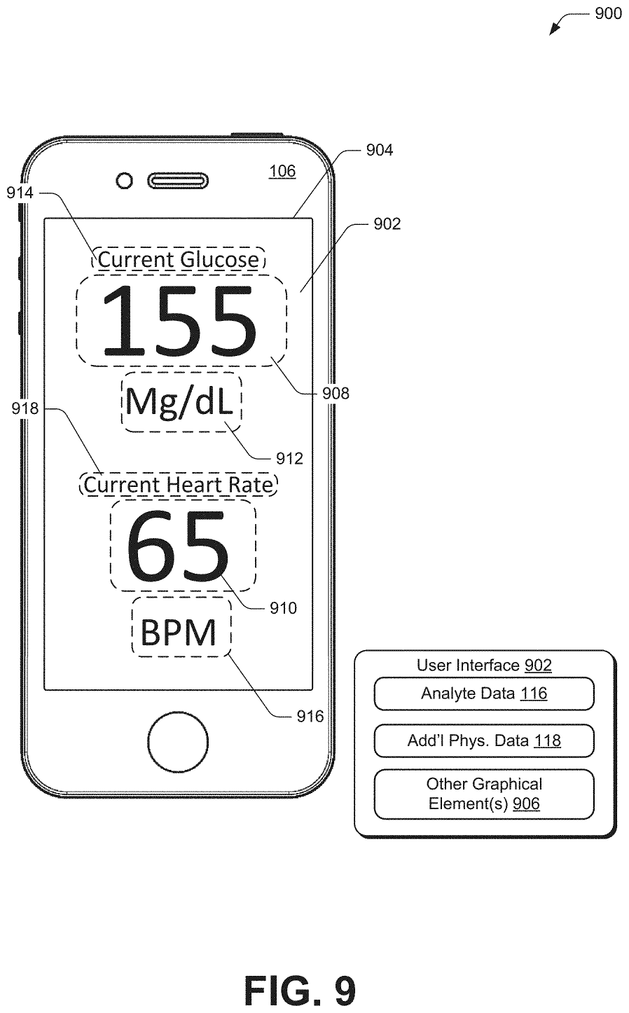

FIG. 9 depicts an example 900 of an implementation of a user interface of computing device 106 displaying both analyte data obtained from a wearable analyte monitoring device and additional physiological data obtained from an analyte augmentation wearable.

The illustrated example 900 depicts the computing device 106 displaying a user interface 902 via a display device 904. Here, the user interface 902 is depicted including both analyte data 116 and additional physiological data 118 that augments the analyte data 116 and other graphical elements 906. In particular, the analyte data 116 displayed via the user interface 902 includes a current glucose 908. The additional physiological data 118, in this example, corresponds to heart rate data that is sensed by one or more sensors of the analyte augmentation wearable 114, and is displayed via the user interface 902 as a current heart rate 910. In one or more implementations, the current glucose 908 and current heart rate 910 are displayed by the user interface 902 in real-time, e.g., as the augmented analyte packet 120 containing both the analyte data 116 and the additional physiological data 118 is received by the sensor hub 122 from one of the wearable analyte monitoring device 112 or the analyte augmentation wearable 114. In this way, the current glucose 908 and current heart rate 910 may correspond to a most recently received analyte measurement and heart rate measurement—the analyte measurement and heart rate measurement most-recently produced by the wearable analyte monitoring device 112 and the analyte augmentation wearable 114, respectively.

In this example, the other graphical elements 906 displayed via the user interface 902 may include a first unit indicator 912 (e.g., “Mg/dL”), a first value label 914, which indicates that the numerical value displayed is a current glucose of a person, a second unit indicator 916 (e.g., “BPM”), and a second value label 918, which indicates that the numerical value displayed is a current heart rate of the person. Notably, the user interface 902 may be configured to include additional information (e.g., a recommendation or insight generated based on the analyte data 116 and/or the additional physiological data 118) along with the analyte data 116 and the additional physiological data 118. The user interface 902 may also be configured to present aggregate metrics, such as “deterioration risk” (e.g., sepsis deterioration risk metric) which may be determined from a combination of the analyte data 116 and/or the additional physiological data 118 and/or determined based on covariance of the signals. An aggregate metric, such as “deterioration risk,” may be presented via the user interface in a variety of ways, such as by using categorical attribute descriptors (e.g., ‘low’, ‘medium’, ‘high’), continuous values (e.g., a score), or a combination of them, to name just a few.

Having discussed exemplary details of the techniques for augmented analyte monitoring systems, consider now some examples of procedures to illustrate additional aspects of the techniques.

### Example Procedures

This section describes examples of procedures for augmented analyte monitoring systems. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by an augmented analyte monitoring system, such as augmented analyte monitoring system 104, by a sensor hub, such as sensor hub 122, and/or by a computing application, such as health monitoring application 126.

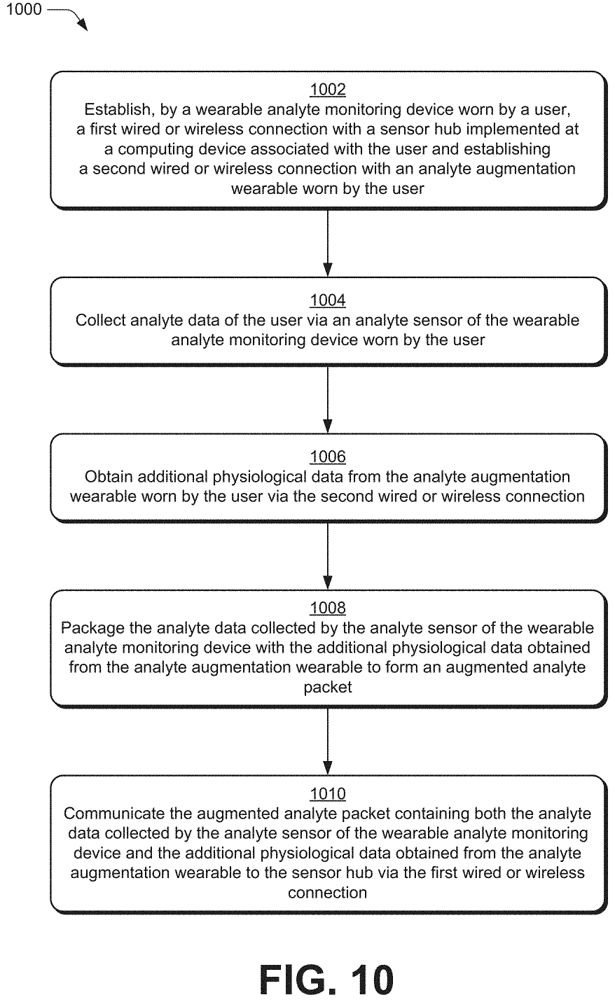

FIG. 10 depicts a procedure 1000 in an example implementation in which a wearable analyte monitoring device generates a data packet containing both analyte data and additional physiological data and communicates the data packet to a sensor hub.

A first wired or wireless connection is established with a sensor hub implemented at a computing device associated with a user and a second wired or wireless connection is established with an analyte augmentation wearable worn by the user (block 1002). By way of example, the wearable analyte monitoring device 112 and the analyte augmentation wearable 114 are communicably coupled via coupling 302. Additionally, the wearable analyte monitoring device 112 and the sensor hub 122 are communicably coupled via coupling 304. The coupling 302 between the wearable analyte monitoring device 112 and the analyte augmentation wearable 114 may be wired (or otherwise a physical coupling of signal transmitting and receiving components) or wireless (including using the body of the person 102 or light) for communicating and signals, examples of these types of couplings are discussed in more detail above. The coupling 304 between the wearable analyte monitoring device 112 and the sensor hub 122 may also be wired or wireless.

Analyte data of the user is collected via an analyte sensor of the wearable analyte monitoring device worn by the user (block 1004). By way of example, the wearable analyte monitoring device 112 may be configured with a sensor that detects signals indicative of the analyte level of the person 102 and enables generation of analyte measurements. Those analyte measurements may correspond to or otherwise be packaged for communication to the computing device 106 as analyte data 116.

Additional physiological data is obtained from an analyte augmentation wearable worn by the user via the second wired or wireless connection (block 1006). By way of example, the wearable analyte monitoring device 112 obtains the additional physiological data 118 from the analyte augmentation wearable 114 via the wired or wireless coupling 302.

The analyte data collected by the analyte sensor of the wearable analyte monitoring device is packaged with the additional physiological data obtained from the analyte augmentation wearable to form an augmented analyte packet (block 1008). By way of example, the wearable analyte monitoring device 112 packages the additional physiological data 118 obtained from the analyte augmentation wearable 114 with the analyte data 116 collected by the analyte sensor to form the augmented analyte packet 120.

The augmented analyte packet containing both the analyte data collected by the analyte sensor of the wearable analyte monitoring device and the additional physiological data obtained from the analyte augmentation wearable is communicated to the sensor hub via the first wired or wireless connection (block 1010). By way of example, the wearable analyte monitoring device 112 transmits the augmented analyte packet 120 containing both the analyte data 116 and the additional physiological data 118 to the sensor hub 122 over the coupling 304. Notably, the wearable analyte monitoring device 112 may communicate the augmented analyte packet 120 data in real-time, e.g., as it is produced using an analyte and/or other sensor. Alternatively or in addition, the analyte monitoring device 112 may communicate the data to the computing device 106 at intervals of time. For example, the wearable analyte monitoring device 112 may be configured to communicate the augmented analyte packets 120 to the computing device 106 approximately every five minutes (as they are being produced).

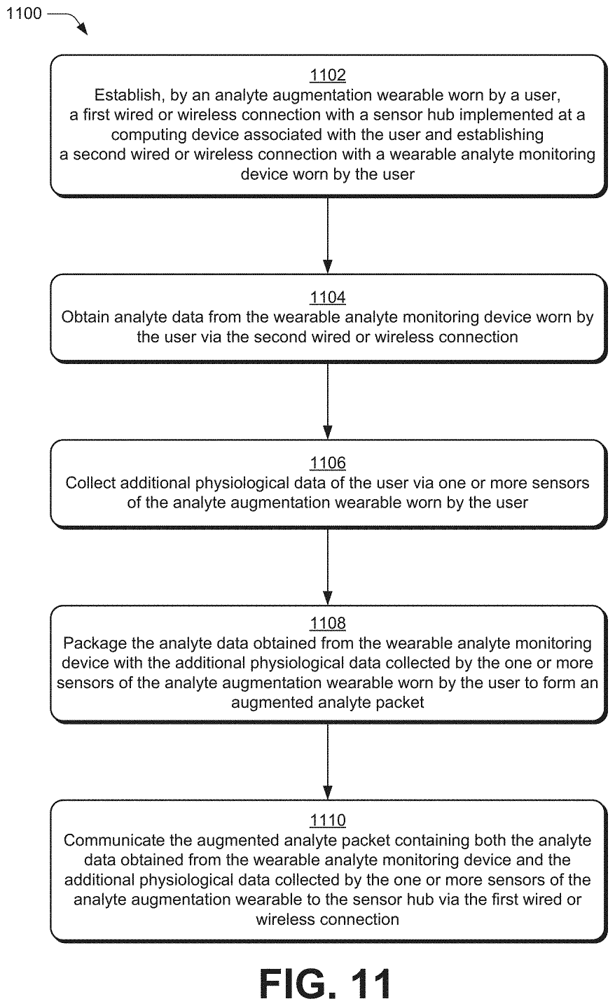

FIG. 11 depicts a procedure 1100 in an example implementation in which an analyte augmentation wearable generates a data packet containing both analyte data and additional physiological data and communicates the data packet to a sensor hub.

A first wired or wireless connection is established with a sensor hub implemented at a computing device associated with a user and a second wired or wireless connection is established with a wearable analyte monitoring device worn by the user (block 1102). By way of example, the wearable analyte monitoring device 112 and the analyte augmentation wearable 114 are communicably coupled via coupling 402. Additionally, the analyte augmentation wearable 114 and the sensor hub 122 are communicably coupled via coupling 404. The coupling 402 between the wearable analyte monitoring device 112 and the analyte augmentation wearable 114 may be wired (or otherwise a physical coupling of signal transmitting and receiving components) or wireless (including using the body of the person 102 or light) for communicating and signals. The coupling 404 between the analyte augmentation wearable 114 and the sensor hub 122 may also be wired or wireless.

Analyte data is obtained from the wearable analyte monitoring device worn by the user via the second wired or wireless connection (block 1104). By way of example, the analyte augmentation wearable 114 obtains the analyte data 116 from the wearable analyte monitoring device 112 via the coupling 402.

Additional physiological data of the user is collected via one or more sensors of the analyte augmentation wearable worn by the user (block 1106). By way of example, the one or more sensors of the analyte augmentation wearable 114 collect additional physiological data 118.

The analyte data obtained from the wearable analyte monitoring device is packaged with the additional physiological data collected by the one or more sensors of the analyte augmentation wearable worn by the user to form an augmented analyte packet (block 1108). By way of example, the analyte augmentation wearable 114 packages the analyte data 116 obtained from the analyte augmentation wearable 114 with the additional physiological data 118 collected by the one or more sensors of the analyte augmentation wearable 114 to form the augmented analyte packet 120.

The augmented analyte packet containing both the analyte data obtained from the wearable analyte monitoring device and the additional physiological data collected by the one or more sensors of the analyte augmentation wearable is communicated to the sensor hub via the first wired or wireless connection (block 1110). By way of example, the analyte augmentation wearable 114 transmits the augmented analyte packet 120 containing both the analyte data 116 and the additional physiological data 118 to the sensor hub 122 over the coupling 404. Notably, the analyte augmentation wearable 114 may communicate the augmented analyte packet 120 in real-time, e.g., as it is produced using an analyte and/or other sensor. Alternatively or in addition, the analyte augmentation wearable 114 may communicate the data to the computing device 106 at intervals of time. For example, the analyte augmentation wearable 114 may be configured to communicate the augmented analyte packets 120 to the computing device 106 approximately every five minutes (as they are being produced).

Having described examples of procedures in accordance with one or more implementations, consider now one example implementation of the techniques described herein.

### Optical Sensing Implementation Example

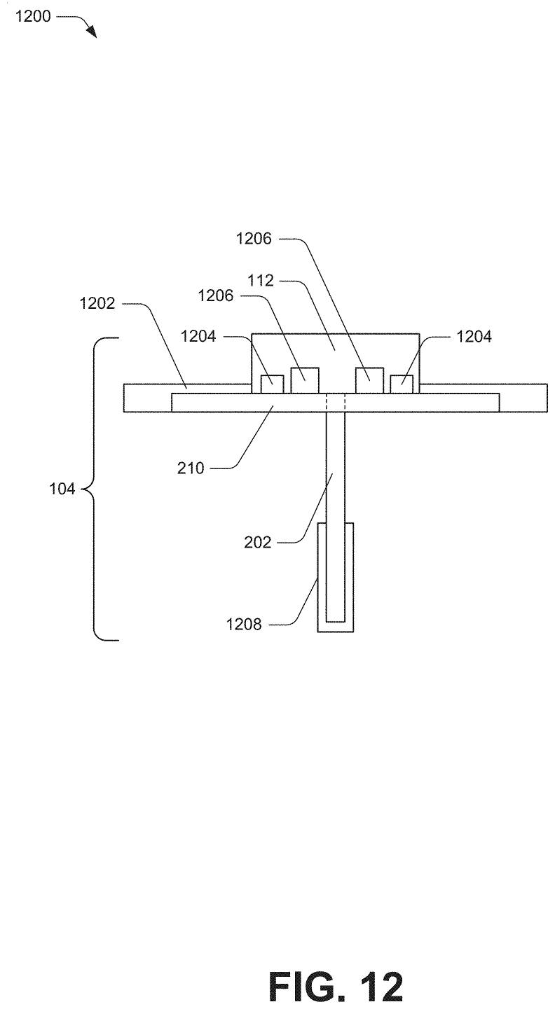

In at least one implementation, the augmented analyte monitoring system 104 includes the wearable analyte monitoring device 112 and an analyte augmentation wearable 114, which is configured at least for optical sensing. Based on one or more optical sensing techniques, the analyte augmentation wearable 114 is configured to produce optical sensing data (e.g., an example of the additional physiological data 118), which is used in various ways to augment the analyte data 116. In at least one variation, such optical sensing data enables the described systems to analyze emissions signals from tissue, such as by using red and/or infrared light to determine or otherwise analyze oxygen saturation (SpO₂) and heart rate measurements of the person 102. Alternatively or in addition, such optical sensing data enables the described systems to analyze emissions from an embedded analyte sensitive dye that is embedded in the analyte augmentation wearable. The analyte augmentation wearable 114 may be configured in a variety of ways to support optical sensing. In the context of optical sensing, consider the following discussion of FIGS. 12-14.

FIG. 12 depicts an example 1200 of the augmented analyte monitoring system that includes an analyte augmentation wearable configured for optical sensing techniques.

The illustrated example 1200 depicts the augmented analyte monitoring system 104, including the wearable analyte monitoring device 112 having the analyte sensor 202 which is insertable into the skin 206 of a host, such as the person 102. The example 1200 also includes the adhesive pad 210. In one or more implementations, the analyte augmentation wearable 104 includes, by way of example and not limitation, one or more of a light filtering component 1202 (configured as a light filtering overlay in this example, a light emitting diode 1204 (LED), a photodetector 1206, and/or an infrared sensor 1208. Analyte augmentation wearable 104 may be configured with various different components without departing from the spirit or scope of the techniques described herein.

In one or more implementations, the light emitting diodes 1204 and the photodetectors 1206 are outside the skin when the augmented analyte monitoring system 104 is deployed. In such implementations, the light source (e.g., one or more light emitting diodes 1204) and the photodetectors 1206 can be integrated into the wearable analyte monitoring device 112 on a skin-facing surface of the wearable analyte monitoring device 112, i.e., underneath the wearable analyte monitoring device 112. Based on excitation and emission, the analyte augmentation wearable 114 enables non-invasive metrics to be measured using one or more algorithms. In variations and using data produced by the analyte augmentation wearable 114, such non-invasive metrics may be measured by one or more of the wearable analyte monitoring device 112, the analyte augmentation wearable 114, the augmented analyte monitoring system 104, and/or the computing device 106 (or various components of the computing device 106). Examples of such metrics include, but are not limited to, heart rate, photoplethysmography (PPG), oxygen saturation (SpO₂), resting heart rate, blood pressure, and any of the other metrics described above or below.

Returning to the illustrated example 1200, it depicts two light emitting diodes 1204 and two photodetectors 1206 incorporated into the wearable analyte monitoring device 112. In one or more implementations, the light emitting diodes 1204 are configured to flash and the photodetectors 1206 are configured to detect emission from tissue (e.g., skin) that is attributed to each light emitting diode 1204. The system then analyses signals produced by the photodetectors 1206 based on the detected emissions using one or more algorithms, e.g., to produce one or more of the above-noted measurements. In at least one variation, the photodetectors 1206 are coated with an optical filter to allow in light having wavelengths within a range (e.g., only allow in light within the wavelength range) for the analysis. In at least one variation, the light filtering component 1202 is an adhesive light filtering overlay. In at least one variation, the light filtering component 1202 is configured to block background light that may interfere with the emission and detection of light by the light emitting diodes 1204 and the photodetectors 1206. Optionally, an augmented analyte monitoring system 104 configured with an analyte augmentation wearable 114 for optical sensing does not include a light filtering component 1202. In the context of analyte augmentation wearables 114 that are differently configured for optical sensing, consider the following examples.

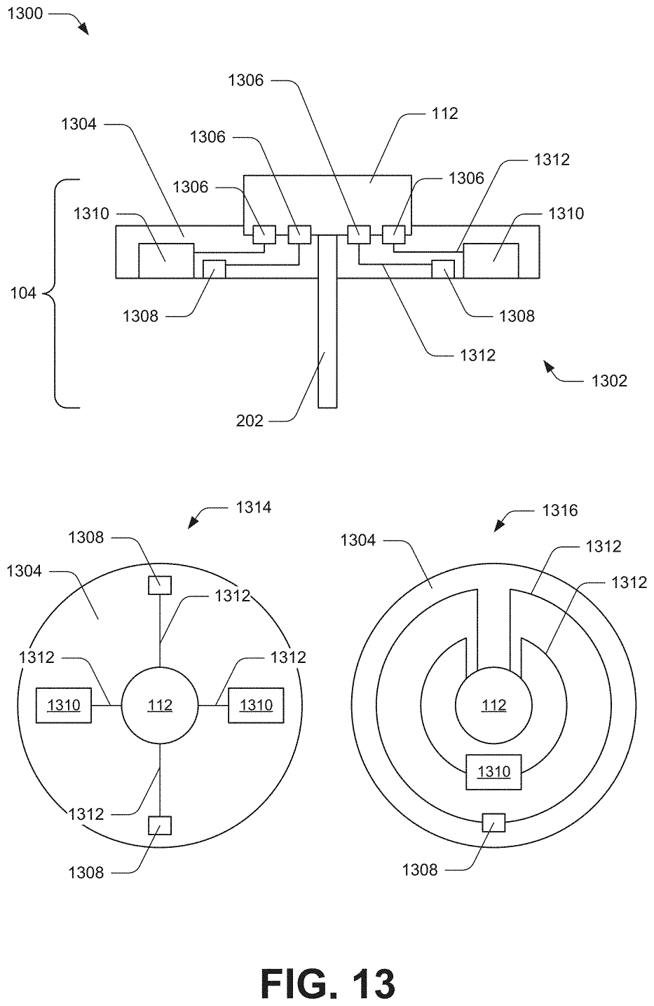

FIG. 13 depicts an example 1300 of the augmented analyte monitoring system that includes an analyte augmentation wearable configured as an underlay for optical sensing techniques.

The illustrated example 1300 depicts a first view 1302 of the augmented analyte monitoring system 104, which is a cutaway side view of the system. In this example, the first view 1302 includes the wearable analyte monitoring device 112 having the analyte sensor 202 which is insertable into the skin 206 of a host, such as the person 102. In this example 1300, the augmented analyte monitoring system 104 includes one or more of a light filtering adhesive component 1304 (configured as a light filtering underlay in this example), electrical pins 1306, light emitting diodes 1308 (LEDs), photodetectors 1310, and traces 1312 connecting the electrical pins 1306 to the light emitting diodes 1308 and the photodetectors 1310. Although not depicted, the analyte augmentation wearable 114 includes one or more infrared sensors in variations.

As an underlay, the light filtering adhesive component 1304 is positioned between skin of a host on which the augmented analyte monitoring system 104 is deployed and the wearable analyte monitoring device 112. In the depicted configuration, the electrical pins 1306 are integral with a skin-facing side (or surface) of the wearable analyte monitoring device 112.

The illustrated example 1300 also depicts a first configuration 1314 and a second configuration 1316 of geometries of the traces 1312, in accordance with one or more variations. In the first configuration 1314, the traces 1312 extend radially from the wearable analyte monitoring device 112 within or on a surface of the light filtering adhesive component 1304 to the light emitting diodes 1308 and the photodetectors 1310. In the second configuration 1316, the traces extend from the wearable analyte monitoring device 112 and partially form circular shapes around the wearable analyte monitoring device 112 while connecting to the light emitting diodes 1308 and the photodetectors 1310. It is to be appreciated that the first configuration 1314 and the second configuration 1316 are merely examples of how the traces 1312 may be incorporated within or on a surface of an analyte augmentation wearable to connect the wearable analyte monitoring device 112 to one or more components (optical or otherwise), e.g., light emitting diodes 1308, photodetectors 1310, and so forth. In variations, the traces 1312 may be configured in other ways, such as having a spiral or ring shape. In one or more implementations, the traces 1312 are configured to carry one or more of power and/or signal between the wearable analyte monitoring device 112 and the components (e.g., the light emitting diodes 1308 and the photodetectors 1310). In one or more implementations, the optical components are incorporated on a surface of an overlay or underlay, however, in variations the optical components are incorporated inside such overlays or underlays.

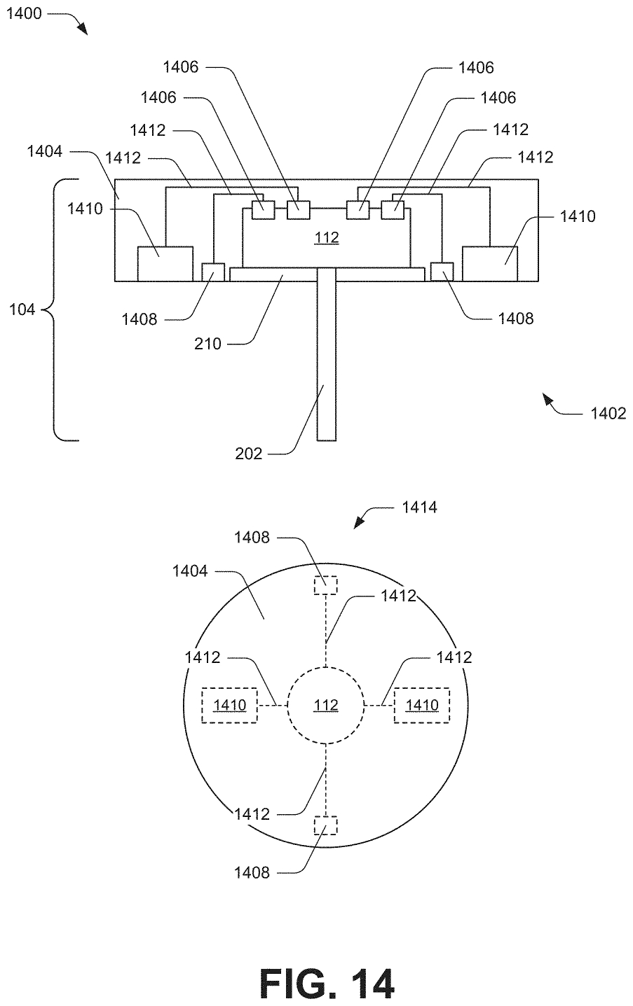

FIG. 14 depicts an example 1400 of the augmented analyte monitoring system that includes an analyte augmentation wearable configured as an overlay for optical sensing techniques.

The illustrated example 1400 depicts a first view 1402 of the augmented analyte monitoring system 104, which is a cutaway side view of the system. In this example, the first view 1402 includes the wearable analyte monitoring device 112 having the analyte sensor 202 which is insertable into the skin 206 of a host, such as the person 102. In this example 1400, the augmented analyte monitoring system 104 includes one or more of a light filtering component 1404 (configured as a light filtering overlay in this example), electrical pins 1406, light emitting diodes 1408 (LEDs), photodetectors 1410, and traces 1412 connecting the electrical pins 1406 to the light emitting diodes 1408 and the photodetectors 1410. Although not depicted, the analyte augmentation wearable 114 includes one or more infrared sensors in variations, e.g., integral with the analyte sensor 202.

As an overlay, the light filtering component 1404 is positioned “on top” of the wearable analyte monitoring device 112, such that the wearable analyte monitoring device 112 is positioned substantially between skin of a host on which the augmented analyte monitoring system 104 is deployed and the light filtering component 1404. In the depicted configuration, the electrical pins 1306 are integral with a top side (or surface) of the wearable analyte monitoring device 112.

The illustrated example 1400 also depicts a configuration 1414 of a geometry of the traces 1412, in accordance with one or more variations. In the depicted configuration 1414, the traces 1412 extend radially from the wearable analyte monitoring device 112 within or on a surface of the light filtering component 1404 to the light emitting diodes 1408 and the photodetectors 1410. The wearable analyte monitoring device 112, the light emitting diodes 1408, the photodetectors 1410, and the traces 1412 are depicted with dashed lines in the illustrated example 1400 to indicate that the light filtering component 1404 may cover those components when deployed. In other words, the configuration 1414 may correspond to a top down view of the augmented analyte monitoring system 104 when deployed with an overlay patch. As noted above, traces (e.g., the traces 1412) may be configured in various ways (e.g., various shapes) to connect the wearable analyte monitoring device 112 to the light emitting diodes 1408 and the photodetectors 1410 without departing from the spirit or scope of the described techniques.

In implementations, such as the example 1400, placement of the light filtering component 1404 on the wearable analyte monitoring device 112 results in establishing electrical connections between the traces 1412 and the electrical pins 1406. In one or more implementations, configuration of the traces 1412 in a spiral or ring geometry can make establishing such an electrical connection easier. In one or more variations, the augmented analyte monitoring system 104 can include multiple sets of LEDs and photodetectors, such as to use different sets to measure multiple analytes (e.g., glucose and lactate) optically and/or to measure multiple non-invasive metrics optically, such as one or more of those described above.

Regarding the incorporation of analyte sensitive dyes, in one or more implementations, one or more of the wearable analyte monitoring device 112 or the analyte augmentation wearable 114 may be configured with analyte sensitive dyes (e.g., embedded in them). Such dyes can be excited at certain wavelengths of light emitted by LEDs of the system and emit light at a particular different wavelength of light detectable by photodetectors of the system. These wavelengths can be changed by using different types of optical dyes. Responsive to exposure to one or more analytes of interest, the dyes undergo changes in characteristics (e.g., chemical changes) that result in a change in signal intensity (e.g., as detected by the photodetectors), change of signal emission time (e.g., as detected by the photodetectors a period of time after the LEDs emit light), and changes in emission wavelength (e.g., as detected by the photodetectors). In one or more implementations, such changes can correlate with a concentration of an analyte of interest, which is determinable by the system. In one or more implementations, the one or more dyes include an oxygen sensitive dye, which changes characteristics of its emission after excitation reflects a level of surrounding oxygen.

Oxygen sensitive dyes may be used because oxidoreductase enzymes consume oxygen to catalyze certain analytes. During such reactions, a biosensor with incorporated oxidoreductase can measure a concentration of enzyme hydrogen peroxide using electrochemical techniques. Changes in oxygen can be recorded with optical techniques using optical dyes as described above. Notably, there are numerous different dyes that can be used to measure different analytes and enzymatic reactions, some examples of these dyes have optical sensitivity to changes in concentration of oxygen, carbon dioxide, pH+, NH₃, etc. Additionally, a dye can be chosen to match the wavelength of excitation from the LEDs used for noninvasive sensing of PPG, Hr, SPO₂, and so on. For example, use of optical sensitive dyes that are excited with red light matches the wavelength of PPG measurements.

In one or more implementations, an architecture for using optical dyes leverages the coaxial geometry of wire of the analyte sensor 202, in which optical dye is incorporated into an EZL and/or RL membrane. In such implementations, one or more LEDs and/or photodetectors may be placed outside the skin in various configurations. Alternatively or additionally, optical techniques may leverage a planner sensor architecture in which LEDs are mounted on top of a planner substrate and connected to the wearable analyte monitoring device 112 with electrical traces. In such examples, working electrodes (WE) can be used for electrochemical sensing while LEDs mounted on circuits can be used for optical sensing. Notably, in such cases in which the LEDs are inside a host, an EZL needs to be deposited on top of the LEDs. In one or more implementations, this process is similar to depositing EZL on a planner electrode, although with the modification of having an optical sensitive dye mixed inside the polymer. In one or more variations, a photodetector can be inside and over a substrate and next to the LEDs and electrochemical electrodes, while in some other cases the photodetector can be on the outside, and over the skin to read the emission and excitation.

Having described at least one implementation example, consider now an example of a system and device that can be utilized to implement the various techniques described herein.

### Example System and Device

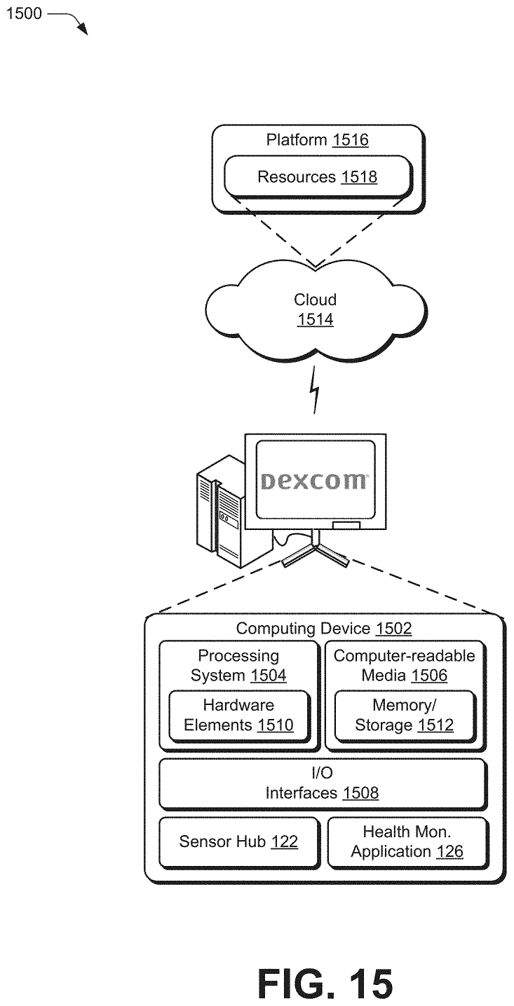

FIG. 15 illustrates an example of a system 1500 generally that includes an example of a computing device 1502 that is representative of one or more computing systems and/or devices that may implement the various techniques described herein. This is illustrated through inclusion of the sensor hub 122 and the health monitoring application 126. The computing device 1502 may be, for example, a server of a service provider, a device associated with a client (e.g., a client device), an on-chip system, and/or any other suitable computing device or computing system.

The example computing device 1502 as illustrated includes a processing system 1504, one or more computer-readable media 1506, and one or more I/O interfaces 1508 that are communicably coupled, one to another. Although not shown, the computing device 1502 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1504 is representative of functionality to perform one or more operations using hardware. Accordingly, the processing system 1504 is illustrated as including hardware elements 1510 that may be configured as processors, functional blocks, and so forth. This may include implementation in hardware as an application specific integrated circuit or other logic device formed using one or more semiconductors. The hardware elements 1510 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1506 is illustrated as including memory/storage 1512. The memory/storage 1512 represents memory/storage capacity associated with one or more computer-readable media. The memory/storage 1512 may include volatile media (such as random access memory (RAM)) and/or nonvolatile media (such as read only memory (ROM), Flash memory, optical disks, magnetic disks, and so forth). The memory/storage 1512 may include fixed media (e.g., RAM, ROM, a fixed hard drive, and so on) as well as removable media (e.g., Flash memory, a removable hard drive, an optical disc, and so forth). The computer-readable media 1506 may be configured in a variety of other ways as further described below.

Input/output interface(s) 1508 are representative of functionality to allow a user to enter commands and information to computing device 1502, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1502 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1502. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1502, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1510 and computer-readable media 1506 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1510. The computing device 1502 may be configured to implement particular instructions and/or functions corresponding to the software and/or hardware modules. Accordingly, implementation of a module that is executable by the computing device 1502 as software may be achieved at least partially in hardware, e.g., through use of computer-readable storage media and/or hardware elements 1510 of the processing system 1504. The instructions and/or functions may be executable/operable by one or more articles of manufacture (for example, one or more computing devices 1502 and/or processing systems 1504) to implement techniques, modules, and examples described herein.

The techniques described herein may be supported by various configurations of the computing device 1502 and are not limited to the specific examples of the techniques described herein. This functionality may also be implemented all or in part through use of a distributed system, such as over a “cloud” 1514 via a platform 1516 as described below.

The cloud 1514 includes and/or is representative of a platform 1516 for resources 1518. The platform 1516 abstracts underlying functionality of hardware (e.g., servers) and software resources of the cloud 1514. The resources 1518 may include applications and/or data that can be utilized while computer processing is executed on servers that are remote from the computing device 1502. Resources 1518 can also include services provided over the Internet and/or through a subscriber network, such as a cellular or Wi-Fi network.

The platform 1516 may abstract resources and functions to connect the computing device 1502 with other computing devices. The platform 1516 may also serve to abstract scaling of resources to provide a corresponding level of scale to encountered demand for the resources 1518 that are implemented via the platform 1516. Accordingly, in an interconnected device embodiment, implementation of functionality described herein may be distributed throughout the system 1500. For example, the functionality may be implemented in part on the computing device 1502 as well as via the platform 1516 that abstracts the functionality of the cloud 1514.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A system comprising:
a wearable analyte monitoring device comprising a transmitter and an analyte sensor to obtain analyte data of a user;
an analyte augmentation wearable comprising one or more sensors to obtain additional physiological data for augmenting the analyte data of the user, the analyte augmentation wearable communicably coupled to the wearable analyte monitoring device via a wired or wireless connection; and
a sensor hub implemented at a computing device to obtain a data packet containing both the analyte data and the additional physiological data from at least one of the wearable analyte monitoring device or the analyte augmentation wearable, and augment the analyte data by storing the analyte data in association with the additional physiological data.

2. The system of claim 1, wherein the additional physiological data describes at least one of an additional analyte of the user or one or more physiological signals of the user.

3. The system of claim 1, wherein the analyte augmentation wearable has a first form factor that is complementary to a second form factor of the wearable analyte monitoring device.

4. The system of claim 3, wherein the analyte augmentation wearable includes at least one of:
an access that allows the analyte sensor of the wearable analyte monitoring device to pass through the access and into skin of the user;
an access that fits around the wearable analyte monitoring device such that the analyte augmentation wearable can be applied to skin of the user around the wearable analyte monitoring device;
a cavity having a complementary shape to the wearable analyte monitoring device such that the wearable analyte monitoring device fits within the cavity of the analyte augmentation wearable and is covered when applied to the skin of the user; or
a partial cavity having a complementary shape to the wearable analyte monitoring device such that a portion of the wearable analyte monitoring device fits within the partial cavity of the analyte augmentation wearable and such that the portion of the wearable analyte monitoring device is covered when applied while another portion of the wearable analyte monitoring device is exposed.

5. The system of claim 3, wherein the analyte augmentation wearable includes one or more components that physically contact at least a portion of the wearable analyte monitoring device when the analyte augmentation wearable and the wearable analyte monitoring device are worn by the user.

6. The system of claim 1, wherein the analyte augmentation wearable comprises an underlay patch that is configured to be disposed at least partially between the wearable analyte monitoring device and skin of the user.

7. The system of claim 1, wherein the analyte augmentation wearable comprises an overlay patch, and wherein the wearable analyte monitoring device is configured to be disposed at least partially between the analyte augmentation wearable and skin of the user.

8. The system of claim 1, wherein the analyte augmentation wearable comprises an overlay patch with a satellite extension, and wherein the satellite extension is configured to position the one or more sensors of the analyte augmentation wearable at least a threshold distance away from the wearable analyte monitoring device.

9. The system of claim 1, wherein the wearable analyte monitoring device is further configured to:
obtain the additional physiological data from the analyte augmentation wearable via the wired or wireless connection;
form the data packet containing both the analyte data and the additional physiological data; and
transmit the data packet containing both the analyte data and the additional physiological data to the sensor hub using the transmitter.

10. The system of claim 1, wherein the analyte augmentation wearable is further configured to compress the additional physiological data and transmit compressed additional physiological data to the wearable analyte monitoring device.

11. The system of claim 1, wherein the wearable analyte monitoring device is further configured to transmit the analyte data to the analyte augmentation wearable using the transmitter.

12. The system of claim 11, wherein the analyte augmentation wearable is further configured to:
obtain the analyte data from the wearable analyte monitoring device via the wired or wireless connection;
form the data packet containing both the analyte data and the additional physiological data; and
transmit the data packet containing both the analyte data and the additional physiological data to the sensor hub.

13. The system of claim 1, wherein the wearable analyte monitoring device is further configured to compress the analyte data and transmit compressed analyte data to the analyte augmentation wearable.

14. A computer-implemented method comprising:
obtaining analyte data of a user by an analyte sensor of a wearable analyte monitoring device worn by the user;
obtaining additional physiological data for augmenting the analyte data of the user by one or more sensors of an analyte augmentation wearable, the analyte augmentation wearable communicably coupled to the wearable analyte monitoring device via a wired or wireless connection;
obtaining, by a sensor hub implemented at a computing device, a data packet containing both the analyte data and the additional physiological data from at least one of the wearable analyte monitoring device or the analyte augmentation wearable; and
augmenting the analyte data by storing the analyte data in association with the additional physiological data.

15. The computer-implemented method of claim 14, wherein the analyte augmentation wearable has a first form factor that is complementary to a second form factor of the wearable analyte monitoring device.

16. The computer-implemented method of claim 14, further comprising:
obtaining, by the wearable analyte monitoring device, the additional physiological data from the analyte augmentation wearable via the wired or wireless connection;
forming the data packet containing both the analyte data and the additional physiological data; and
transmitting the data packet containing both the analyte data and the additional physiological data to the sensor hub using a transmitter of the wearable analyte monitoring device.

17. The computer-implemented method of claim 14, further comprising:
obtaining, by the analyte augmentation wearable, the analyte data from the wearable analyte monitoring device via the wired or wireless connection;
forming the data packet containing both the analyte data and the additional physiological data; and
transmit the data packet containing both the analyte data and the additional physiological data to the sensor hub using a transmitter of the analyte augmentation wearable.

18. A method implemented by a wearable analyte monitoring device worn by a user, the method comprising:
establishing a first wired or wireless connection with a sensor hub implemented at a computing device associated with the user and establishing a second wired or wireless connection with an analyte augmentation wearable worn by the user;
collecting analyte data of the user via an analyte sensor of the wearable analyte monitoring device worn by the user;
obtaining additional physiological data from the analyte augmentation wearable worn by the user via the second wired or wireless connection;
packaging the analyte data collected by the analyte sensor of the wearable analyte monitoring device with the additional physiological data obtained from the analyte augmentation wearable to form an augmented analyte packet; and
communicating the augmented analyte packet containing both the analyte data collected by the analyte sensor of the wearable analyte monitoring device and the additional physiological data obtained from the analyte augmentation wearable to the sensor hub via the first wired or wireless connection.

19. The method of claim 18, wherein the communicating further comprises communicating the augmented analyte packet containing both the analyte data and the additional physiological data to the sensor hub at predefined intervals.

20. A method implemented by an analyte augmentation wearable worn by a user, the method comprising:
establishing a first wired or wireless connection with a sensor hub implemented at a computing device associated with the user and establishing a second wired or wireless connection with a wearable analyte monitoring device worn by the user;
obtaining analyte data from the wearable analyte monitoring device worn by the user via the second wired or wireless connection;
collecting additional physiological data of the user via one or more sensors of the analyte augmentation wearable worn by the user;
packaging the analyte data obtained from the wearable analyte monitoring device with the additional physiological data collected by the one or more sensors of the analyte augmentation wearable worn by the user to form an augmented analyte packet; and
communicating the augmented analyte packet containing both the analyte data obtained from the wearable analyte monitoring device and the additional physiological data collected by the one or more sensors of the analyte augmentation wearable to the sensor hub via the first wired or wireless connection.

21. The method of claim 20, wherein the communicating further comprises communicating the augmented analyte packet containing both the analyte data and the additional physiological data to the sensor hub at predefined intervals.

22. An apparatus comprising:
one or more sensors to collect physiological data of a user; and
an underlay patch configured to directly contact skin of the user, the underlay patch comprising an access portion;
wherein a wearable analyte monitoring device is configured to be disposed on top of the underlay patch, and wherein the access portion of the underlay patch enables an analyte sensor of the wearable analyte monitoring device to extend through the access portion of the underlay patch and insert subcutaneously into the skin of the user to collect analyte data of the user.

23. The apparatus of claim 22, wherein the one or more sensors comprise at least one of electrodes or photonics.

24. The apparatus of claim 22, wherein the apparatus is configured to communicate the physiological data of the user to the wearable analyte monitoring device via a wired or wireless connection with the wearable analyte monitoring device.

25. An apparatus comprising:
one or more sensors to collect physiological data of a user; and
an overlay patch configured to be applied on top of a wearable analyte monitoring device worn by the user, wherein the overlay patch includes an adhesive for adhering the overlay patch to the wearable analyte monitoring device and skin of the user and wherein the overlay patch has a geometry that is complementary with a form factor of the wearable analyte monitoring device.

26. The apparatus of claim 25, wherein the overlay patch causes the one or more sensors to be deployed within a threshold distance of an analyte sensor of the wearable analyte monitoring device.

27. An apparatus comprising:
one or more sensors to collect physiological data of a user; and
an overlay patch configured to be applied on top of a wearable analyte monitoring device worn by the user, the overlay patch including a satellite extension to position the one or more sensors at least a threshold distance away from an analyte sensor of the wearable analyte monitoring device.

28. The apparatus of claim 27, wherein the overlay patch further includes an adhesive for adhering the overlay patch to the wearable analyte monitoring device and skin of the user.
